(19) United States
(12) Patent Application Publication
(10) Pub. No.: US 2013/0164387 A9
(43) Pub. Date: Jun. 27, 2013

(54) NOVEL MANNOPYRANOSIDE DERIVATIVES WITH ANTICANCER ACTIVITY

(71) Applicants: Jean-Louis Montero, Lauret (FR); Veronique Montero, Lauret (FR); Jean-Pierre Moles, Cournonsec (FR); Pascal De Santa Barbara, Fabregues (FR); Stephanie Combemale, Montpellier (FR); Azzam Awwad, Montpellier (FR); Bernard Jover, Montpellier (FR)

(72) Inventors: Jean-Louis Montero, Lauret (FR); Veronique Montero, Lauret (FR); Jean-Pierre Moles, Cournonsec (FR); Pascal De Santa Barbara, Fabregues (FR); Stephanie Combemale, Montpellier (FR); Azzam Awwad, Montpellier (FR); Bernard Jover, Montpellier (FR)

(73) Assignee: Centre National De La Recherche Scientifique, Paris (FR)

(21) Appl. No.: 13/509,155

(22) PCT Filed: Nov. 10, 2010
(86) PCT No.: PCT/FR10/00749

§ 371 Date: Jul. 10, 2012

(30) Foreign Application Priority Data
Nov. 10, 2009 (FR) 09 05394

(51) Int. Cl. A61K 31/7135; C07H 5/04; A61K 33/24; A61P 27/02; A61P 29/00; A61P 17/06; A61K 31/704; C07H 23/00; A61P 35/00; B82Y 5/00

(52) U.S. Cl. 424/649; 536/17.1; 514/25; 514/34; 977/773; 977/810; 977/906

(57) ABSTRACT

The present invention relates to mannopyranoside-derived compounds and to the use thereof as medicaments, in particular in the treatment of cancer diseases, and also to the method for preparing same and to pharmaceutical compositions comprising such compounds. Medical devices surface-treated with mannopyranoside-derived compounds according to the invention also form part of the invention.

The present invention relates to mannopyranoside-derived compounds, to a process for synthesizing such compounds by green chemistry, and to the use thereof as medicaments, in particular in the treatment of cancer diseases, and also to pharmaceutical compositions comprising such compounds. Implantable medical devices surface-treated with manno-pyranoside-derived compounds according to the invention also form part of the invention.

Numerous pathological conditions have been described in the prior art as having an angiogenesis-related component or stage. Mention may be made, inter alia, of a very large number of cancers, diabetes-related retinopathies, atherosclerosis, arthrosis, rheumatoid arthritis, psoriasis and also inflammatory pathological conditions or pathological conditions associated with delayed wound healing.

Angiogenesis is a mechanism of neovascularization stemming from a preexisting capillary network. The budding of small vessels, the capillaries, from preexisting vessels arises in the best case during embryonic development and implantation of the placenta, when it is the case of healing a wound, or of overcoming the obstruction of a vessel; but also, in the worst case, in cancers (growth of tumors and development of metastases), rheumatoid arthritis, certain ophthalmological diseases such as diabetic retinopathy or age-related macular degeneration. For all these processes, the general scheme remains the same. Activation of the endothelial cells leads to degradation of the basal membrane and of the surrounding extracellular matrix. Directed migration is followed by a proliferative phase. The cells then differentiate into a structure of capillary type so as to form a vascular network necessary for the growth of the tissues. However, angiogenesis is not controlled by a single factor, but by a balance of inducers and inhibitors produced by normal or tumor cells. Among these factors, polypeptides such as fibroblast growth factor-2 (FGF-2) and vascular endothelial growth factor (VEGF) have emerged as being key regulators of angiogenesis.

Many molecules have been studied for their inhibitory or activating effect on angiogenesis.

As regards the inhibition of angiogenesis, a recent conceptual revolution in cancer treatment consists in targeting the vascular network that irrigates a tumor. It is now well established that the development of intratumor or peritumor vascularization is a key event both for the growth of a tumor and for metastatic dissemination via the bloodstream. In December 2005, the British scientific journal Nature, which devoted its issue to angiogenesis, counted more than 300 inhibitors, including 80 undergoing clinical trials. However, the first medicaments tested—angiostatin, endostatin, interferons, matrix metalloproteinase inhibitors, etc.—were disappointing. Among more recent molecules, mention may be made of bevacizumab. When injected into a patient, it neutralizes a type of VEGF circulating in the capillaries or diffuse in the tumor, VEGF-A. Its first indication was in 2004 for metastatic colorectal cancer, in combination with chemotherapy. It is now in clinical trials against metastatic kidney cancer, lung cancer and breast cancer. However, VEGF-A has the drawback of increasing the risk of hypertension and of hemorrhage. Mention may also be made of sunitinib and sorafenib, which have the advantage of being able to allow formulation in the form of tablets to be taken orally and which lead to encouraging therapeutic results. They also have the drawback of giving rise to some side effects such as hypertension, fatigue or skin problems.

Furthermore, the molecules currently used for their angiogenesis-inhibiting properties have high toxicities which are often totally unacceptable in terms of continuing treatments, this toxicity limiting the duration and the efficacy of current medications.

For the purpose of offering novel anticancer treatments that have excellent antitumor activity, there is thus a constant and extremely important need for angiogenesis-inhibiting compounds that have very low toxicity and better affinity to receptors.

The inventors have therefore developed what is the subject of the present invention in order to remedy all these drawbacks and to provide compounds that have angiogenesis-inhibiting activity with very low toxicity allied to excellent activity, it being possible for these compounds in particular to be used for preparing anticancer medicaments.

The inventors have in effect discovered that certain mannopyranoside derivatives have excellent anticancer activity and a very low toxicity, it being possible for these compounds to be used for preparing pharmaceutical compositions intended for the treatment of cancer pathological conditions.

A subject of the present invention is therefore a mannopyranoside-derived compound or pharmaceutically acceptable salts thereof, corresponding to one of the following formulae:

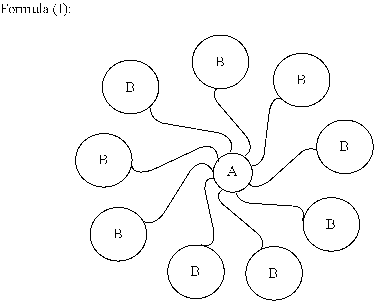

in which:
A is a silicon nanoparticle or a metal nanoparticle chosen from the elements of columns (IB), (IIB), (IIIB), (IVB), (VB), (VIB), (VIIB) or (VIIIB) of Mendeleev's periodic table, and
B is a group carrying a mannopyranoside function, also called “polar head”, corresponding to the following structure:

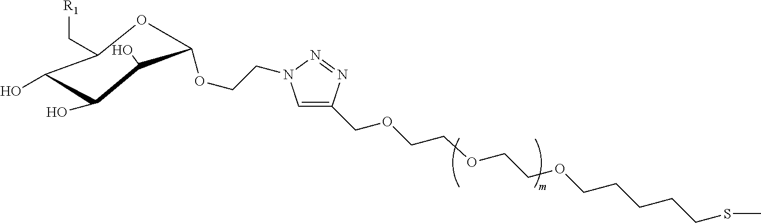

in which m is an integer between 0 and 10, and preferably m=3, 4, 5 or 6,
the B groups being bonded to the nanoparticle A via the sulfur atom, and the number of B groups bonded to the nanoparticle A being between 100 and 1000, and preferably between 400 and 600,

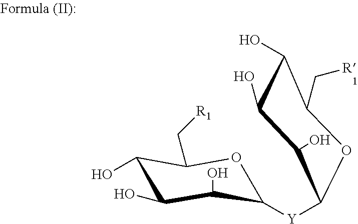

in which Y represents one of the following groups:

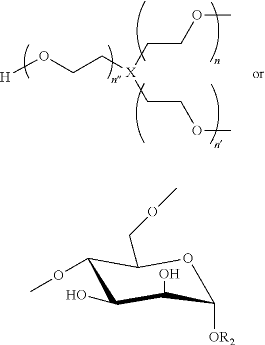

with:
n, n′ and n″ being integers between 1 and 12, and preferably between 1 and 6, and
n″ being equal to 0 when X represents an oxygen atom,

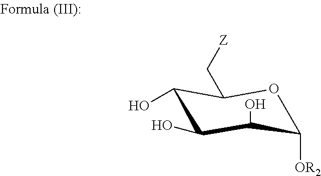

in which Z represents one of the following groups:

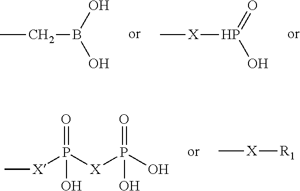

in which:
the R₁ and R′₁ radicals, which may be identical or different, represent a radical selected from —O—PO₃H₂, —N₃, —CH₂—PO₃H₂, —CH₂—COOH, —SO₃H₂, —OPHO₂H, —CH₂—B(OH)₂, —X—PHO₂H, X′—PO₂H—X—PO₃H₂, and preferably —CH₂—COOH and —N₃,
the R₂ radical represents a linear or branched C₁-C₁₂, and preferably C₁-C₄, alkyl chain; a linear or branched C₁-C₁₂, and preferably C₁-C₄, alkyl chain carrying at least one —OH, —NH₂, —SH, —COOH, —N₃ or —NO₂ group; a saturated or unsaturated C₃-C₆ hydrocarbon-based ring; a saturated or unsaturated C₃-C₁₀ hydrocarbon-based ring carrying at least one —OH, —NH₂, —SH, —COOH, —N₃, —NO₂ or C₁-C₄ alkyl group; a saturated or unsaturated heterocycle comprising at least one heteroatom chosen from oxygen, nitrogen or sulfur atoms; a —(CH₂—CH₂—O)_{y}—H radical, in which y is between 1 and 12, and preferably between 1 and 6, and
the X and X′ groups, which may be identical or different, are chosen from: N, O, S and a C₁-C₄ alkyl chain, the X and X′ groups preferably being oxygen atoms.

Among the C₁-C₄ alkyl chains mentioned for R₂, mention may in particular be made of methyl, ethyl, n-propyl, isopropyl, n-butyl, tert-butyl, isobutyl and n-hexyl radicals, the methyl radical being the most preferred.

Among the saturated hydrocarbon-based rings mentioned for R₂, mention may in particular be made of cyclopropane, cyclobutane, cyclopentane and cyclohexane.

Among the unsaturated hydrocarbon-based rings and the saturated heterocycles mentioned for R₂, mention may in particular be made of phenyl, oxadiazole, triazole, oxazole, isoxazole, imidazole, thiadiazole, pyrrole, tetrazole, furan, thiophene, pyrazole, pyrazoline, pyrazolidine, thiazole, isothiazole, pyridine, pyrimidine, piperidine, pyran, pyrazine, pyridazine, indole, indazole, benzoxazole, naphthalene, quinoline, quinoxaline, quinazoline, anthracene and acridine rings, phenyl rings being the most preferred.

According to one advantageous embodiment, when the compound of the invention is a compound of formula (I), the nanoparticle A is chosen from gold, iron and cobalt nanoparticles, and more particularly gold nanoparticles. The nanoparticles A can have a diameter of between 2 and 10 nm, and preferably between 4 and 8 nm.

Owing to the presence of multipolar heads (B groups), the compounds of formula (I) have a better affinity with respect to receptors, and consequently improved anti-angiogenic properties compared with the known compounds of the prior art.

According to another advantageous embodiment, when the compound of the invention is a compound of formula (II) in which:

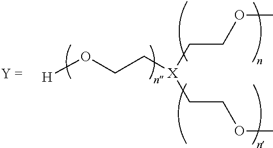

then n=1, n′=2 and n″=0, and X represents an oxygen atom.

The compounds in accordance with the invention can be used as an active ingredient for producing medicaments, in particular for the prevention and/or treatment of diseases dependent on an inhibition of angiogenesis, among which mention may most particularly be made of cancer diseases, diabetic blindness, macular degeneration, rheumatoid arthritis and psoriasis.

Another subject of the invention is a pharmaceutical composition comprising, as an active ingredient, at least one compound according to the invention as defined above, and at least one pharmaceutically acceptable excipient, it being possible for said composition itself also to be used for the prevention and/or treatment of diseases dependent on an inhibition of angiogenesis, such as those mentioned above.

The form of the medicament or of the pharmaceutical composition may be a solution, a suspension, an emulsion, tablets, gel capsules or suppositories, and will depend on the route of administration selected.

Thus, for the purpose of the present invention, the medicament or the pharmaceutical composition can be administered according to any appropriate route, for example orally, locally, systemically, intravenously, intramuscularly or mucosally, or else using a patch.

Depending on the route of administration of the medicament or of the pharmaceutical composition of the invention, those skilled in the art will choose one or more appropriate pharmaceutically acceptable excipients. By way of nonlimiting examples of excipients that are suitable for oral administration, mention may in particular be made of: talc, lactose, starch and its derivatives, cellulose and its derivatives, polyethylene glycols, acrylic acid polymers, gelatin, magnesium stearate, animal, vegetable or synthetic fats, paraffin derivatives, glycols, stabilizers, preservatives, antioxidants, wetting agents, anticaking agents, dispersants, emulsifiers, flavor enhancers, penetrants, solubilizers, etc.

When it is intended for the treatment of cancer diseases, the pharmaceutical composition may also comprise one or more antitumor active ingredients, among which mention may be made of antimitotics, differentiation inducers, antibodies, etc. More particularly, these active ingredients may be doxorubicin, etoposide, fluorouracil, melphalan, cyclophosphamide, bleomycin, vinblastin, mitomycin, lomustine (CCNU), taxotere, taxol, methotrexate and cisplatinum.

The compounds of the invention can be prepared according to processes well known to those skilled in the art, these syntheses having already been described in the following documents:
B. G. Davis et al., J. Org. Chem., 1998, 63, 9614-9615,
M. E. Evans et al., Carb. Res., 1977, 54,105-114,
P. A. M. Van der Klein et al., Carb. Res., 1992, 224, 193-200,
H. H. Baer et al., Carb. Res., 1990, 200, 377-389,
C. Grondal, Synlett, 2003, 10, 1568-1569,
E. A. Hauser et al., Experiments in Colloid Chemistry, McGraw Hill, 1940, p. 18,
J. Turkevich et al., Discuss. Faraday. Soc., 1951, 11, 55-75,
J. Kimling et al., J. Phys. Chem. B, 2006, 110, 15700-15707,
Formation of boronates: R. Soundararajan et al., J. Org. Chem., 1990, 55, 2274-2275,
Formation of borates:
J. Meulenhoff et al., Allg. Chem., 1925, 373,
S. D. Ross et al., J. Org. Chem., 1965, 30, 2852,

C. J. Salomon et al., Tetrahedron Lett., 1995, 36, 6759-6760,
Formation of pyrophosphonates: A. M. Michelson, Biochem. Acta., 1964, 91, 1-13,
Synthesis of hydrogen phosphonates:
Clavel et al., Tetrahedron Letters, 2004, 45(40), 7465-7467,
Z. Ge et al., Journal of Applied Polymer Science, 2007, 104 (2), 1138-1142,
K. Jarowicki et al., Journal of the Chemical Society-Perkin Transactions, 2001, (18), 2109-2135,
F. Onyemauwa et al., Organic Letters, 2006, 8, 5255-5258.

The compounds of the invention can also be prepared according to an ecological process, also known as “green chemistry process”. This process has the advantage of not requiring any solvent, nor any additional step of purification by chromatography, while at the same time making it possible to obtain high yields. Indeed, the processes described in the prior art very often require protection/deprotection steps which consume expensive and polluting reagents and solvents. Thus, the inventors have developed a process for overcoming these drawbacks, said process comprising at least the following steps:

(i) a step of halogenation between a compound of formula (I′), (II′) or (III′) carrying at least one primary alcohol function, by reaction with a dihalogen/phosphine or N-halo-succinimide/phosphine mixture, said compounds (I′), (II′) or (III′) corresponding to the formulae below:

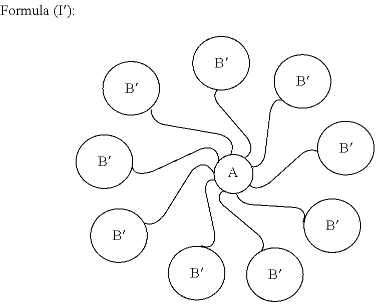

in which A has the same meaning as previously, and B′ is a group corresponding to the following structure:

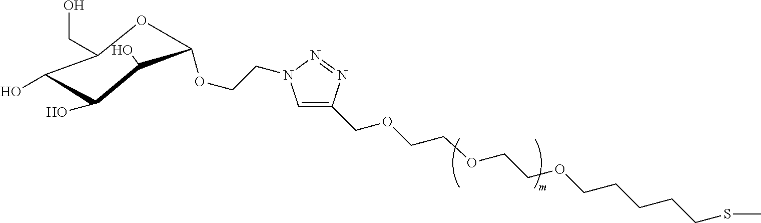

in which m has the same meaning as previously,
the B′ groups being bonded to the nanoparticle A via the sulfur atom, and the number of B′ groups bonded to the nanoparticle A being between 100 and 1000, and preferably between 400 and 600,

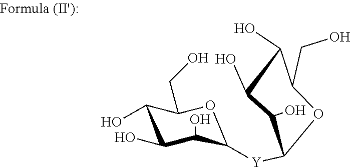

in which Y, n, n′ and n″ have the same meaning as previously,

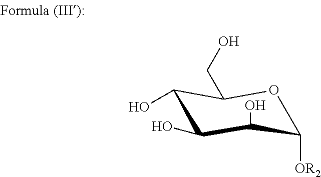

the R₂ radical and the X and X′ groups being as defined previously,

(ii) a step of nucleophilic substitution of the halogenated compounds obtained during step (i), by reaction with a nucleophilic reagent carrying an R₁ and/or R′₁ radical, said nucleophilic reagent preferably being a lithium compound, it being possible in particular for the nucleophilic reagent to be chosen from LiO—PO₃H₂, NaN₃, LiCH₂—PO₃H₂, LiCH₂—COOH, LiSO₃H₂, LiOPHO₂H, LiCH₂—B(OH)₂, LiX—PHO₂H and LiX′—PO₂H—X—PO₃H₂, so as to obtain the compounds of formula (I), (II) or (III) of the invention.

During steps (i) and (ii), the reagents are preferably used in stoichiometric proportions.

According to one advantageous embodiment, the phosphine used in step (i) is chosen from a trialkylphosphine of which the alkyl chain is C₁-C₆, triphenylphosphine Pφ₃ or 2,2′-bis(diphenylphosphino)-1,1′-binaphthyl, and even more preferably step (i) is carried out with an I₂/Pφ₃ mixture, in the presence of imidazole. The reagents used in step (i) are preferably premilled, and then heated in an oil bath at a temperature that can range from 60 to 100° C., with stirring, for a period of between 15 and 30 minutes. At the end of step (i) the reaction mixture is dissolved in a solvent such as methanol, concentrated, and then filtered on silica gel.

Step (ii) can, for its part, advantageously be carried out by mixing the halogenated compound obtained in step (i) with a nucleophilic reagent carrying an R₁ and/or R′₁ radical. The reagents used in step (ii) are preferably premilled, and then heated in an oil bath at a temperature that can range from 60 to 100° C., with stirring, for a period of between 15 and 30 minutes. At the end of step (ii), the reaction mixture is purified, preferably by flash filtration on silica gel.

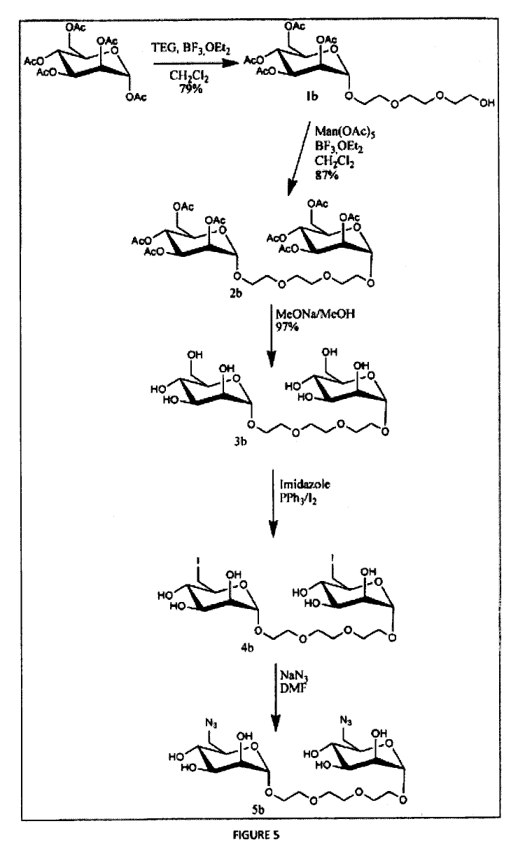

An example of synthesis according to the “green chemistry process” is represented diagrammatically in the appended FIG. 5, the process carried out comprising a step of iodination of α-D-mannopyranoside, the iodo-α-D-mannopyranoside obtained then being reacted in stoichiometric proportions with sodium azide (NaN₃), the latter enabling functionalization of the mannopyranoside in position 6.

Finally, a last subject of the invention relates to a medical device that can be implanted in the human body, said device being surface-treated with at least one mannopyranoside-derived compound according to the invention. Among the devices mentioned above, mention may be made of prostheses, and more particularly vascular, urethral and biliary stents. The need for such devices exists because, currently, many medical devices allow only limited implantation in the human body, owing to excessive angiogenesis.

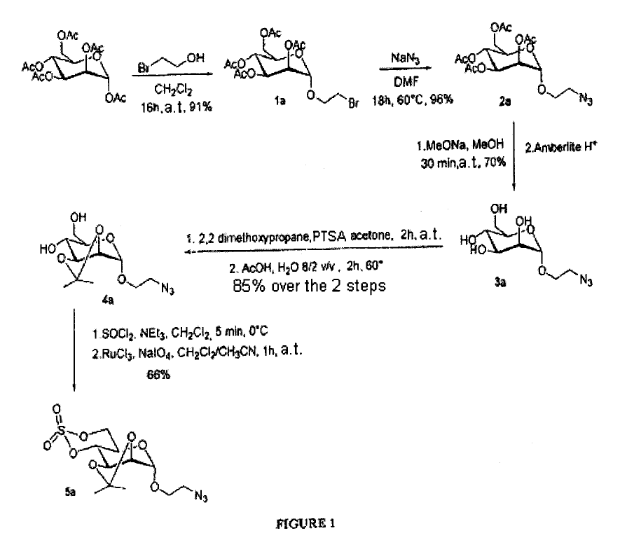

In addition to the above arrangements, the invention also comprises other arrangements which will emerge from the description which follows, which refers to examples of preparation of the compounds in accordance with the invention, and also to examples demonstrating the antitumor activity of the compounds according to the invention compared with other compounds representative of the prior art which do not form part of the invention, and also to the following appended FIGS. 1 to 15:

FIG. 1 represents the scheme for synthesis of the compounds 1a to 5a,

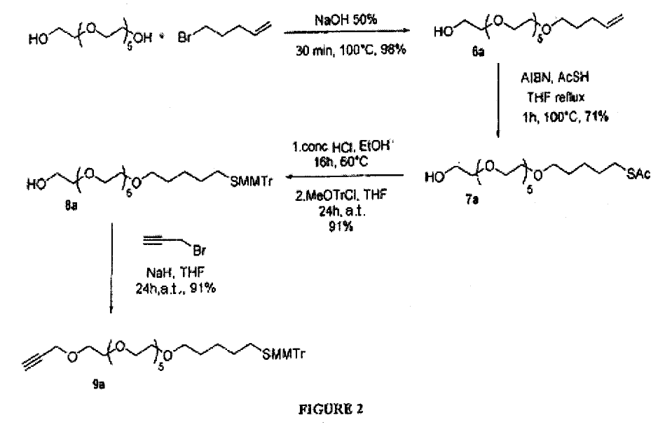

FIG. 2 represents the scheme for synthesis of the compounds 6a to 9a,

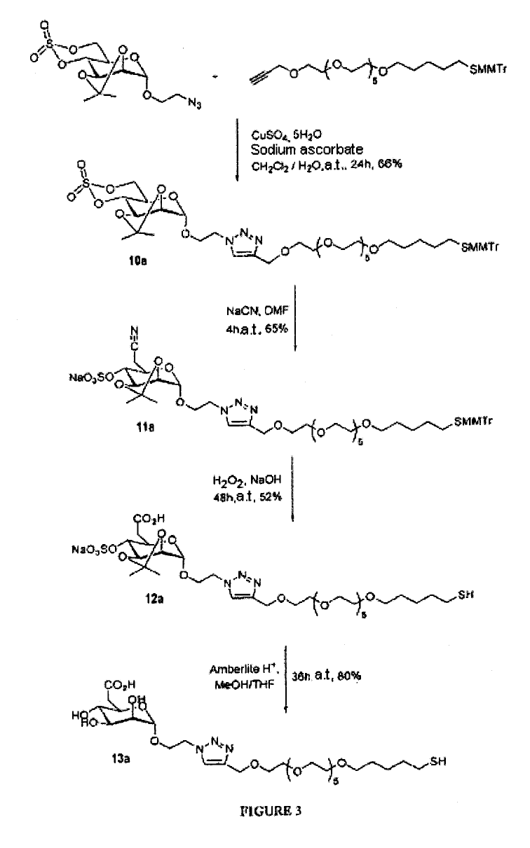

FIG. 3 represents the scheme for synthesis of the compounds 10a to 13a (functionalization of mannopyranoside in position 6 with a carboxy group),

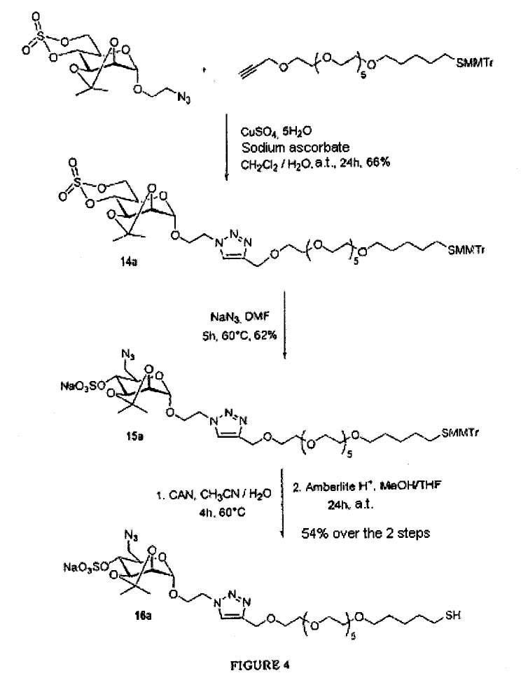

FIG. 4 represents the scheme for synthesis of the compounds 14a to 16a (functionalization of mannopyranoside in position 6 with an azido group),

FIG. 5 represents the scheme for synthesis of a compound of formula (II) according to the invention,

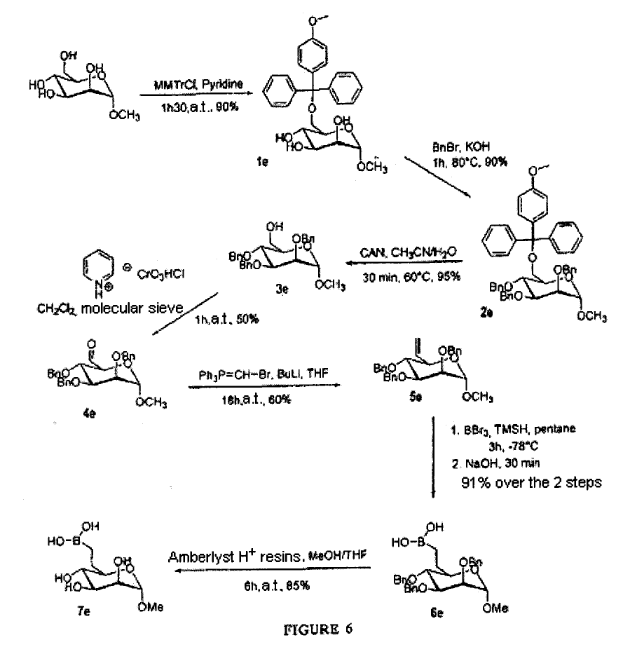

FIG. 6 represents the scheme for synthesis of a compound of formula (III) according to the invention,

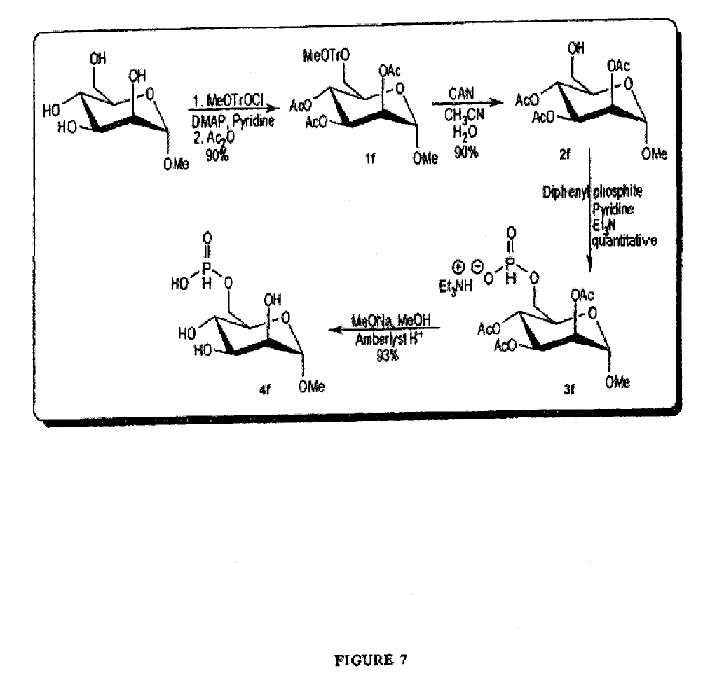

FIG. 7 represents the scheme for synthesis of a compound of formula (III) according to the invention,

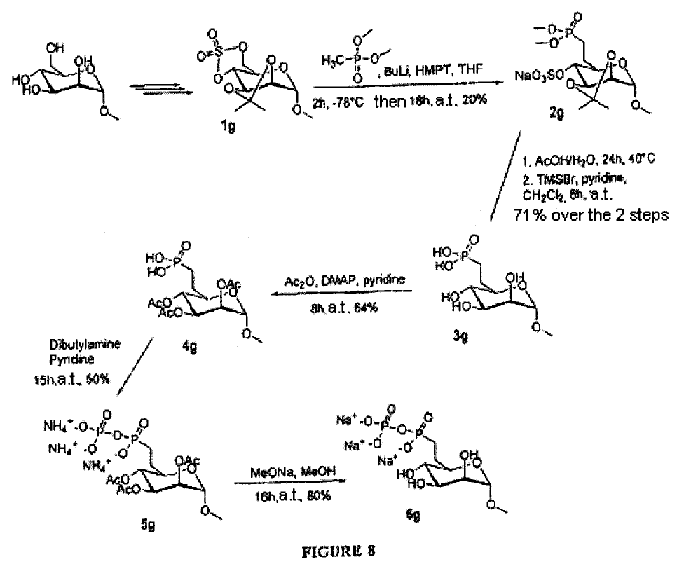

FIG. 8 summarizes the synthesis of a pyrophosphonate derivative (compounds 1g to 6g) corresponding to formula (III) of the invention,

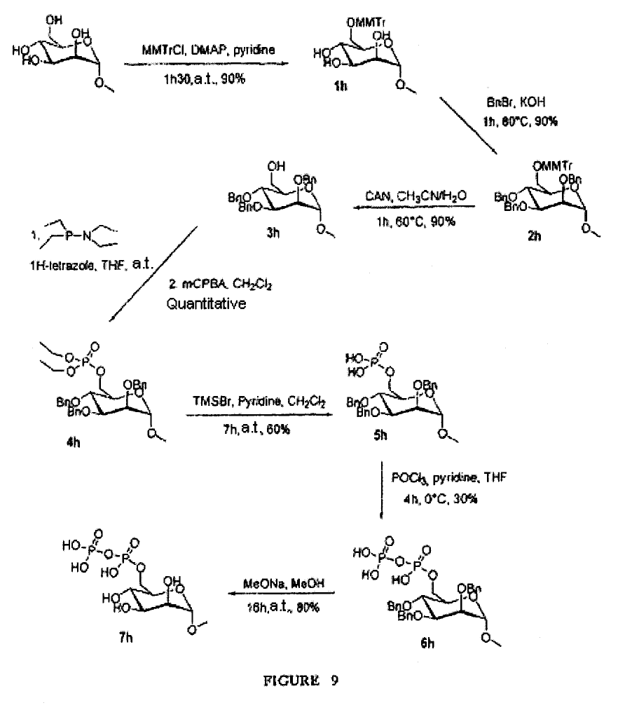

FIG. 9 summarizes the synthesis of a pyrophosphate derivative (compounds 1h to 6h) corresponding to formula (III) of the invention,

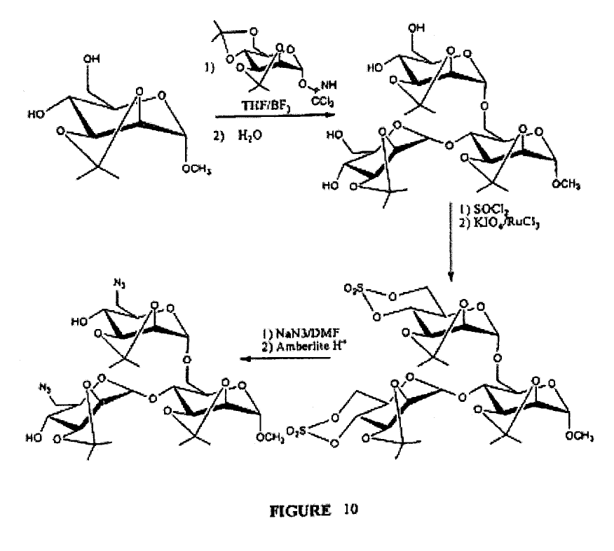

FIG. 10 represents the scheme for synthesis of a compound corresponding to formula (II) of the invention,

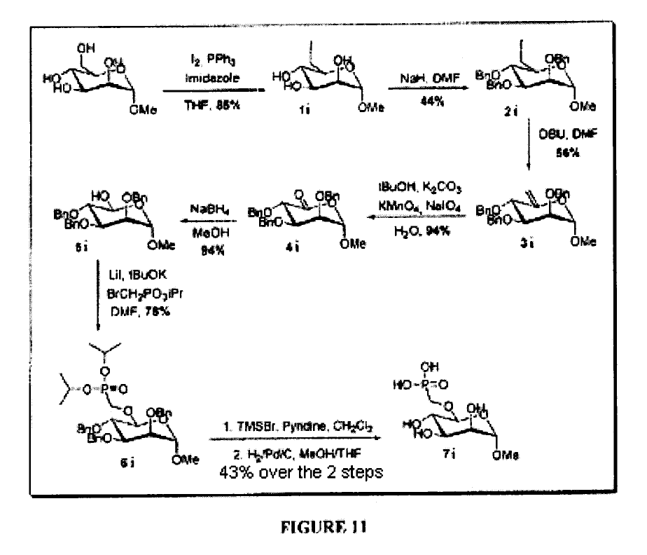

FIG. 11 represents the scheme for synthesis of a compound corresponding to formula (III) of the invention (compounds 1i to 7i),

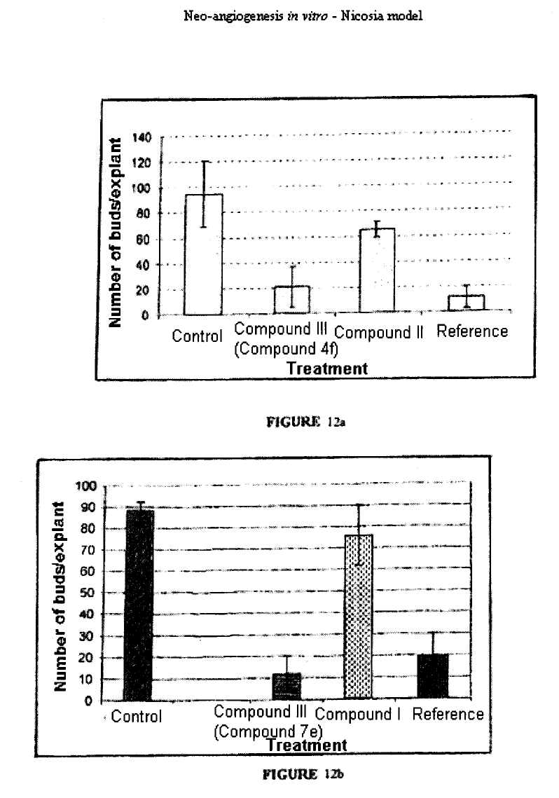

FIGS. 12a and 12b are histograms representing the in vitro neo-angiogenic effect of various compounds of the invention (Nicosia model),

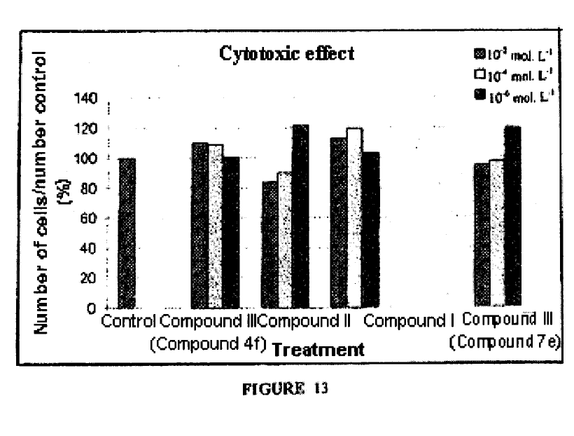

FIG. 13 is a histogram showing the cytotoxic effect of various compounds of the invention,

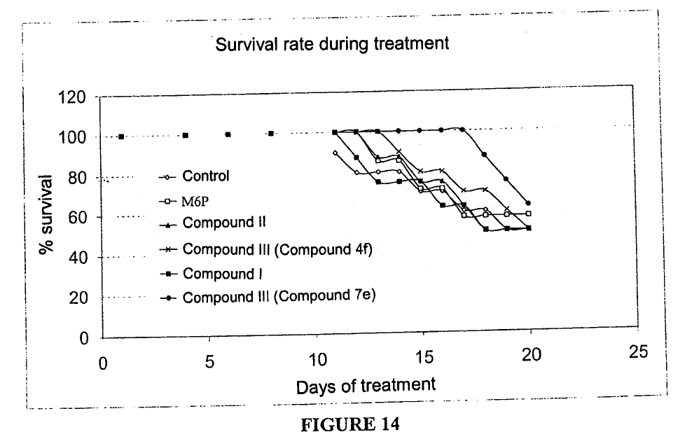

FIG. 14 is a graph representing the survival rate of mice as a function of the number of days of treatment, for various compounds of the invention,

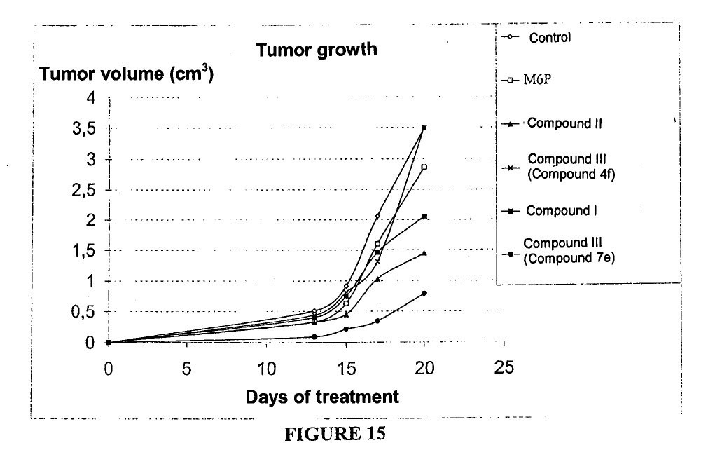

FIG. 15 is a graph representing tumor growth as a function of the number of days of treatment, for various compounds of the invention, and

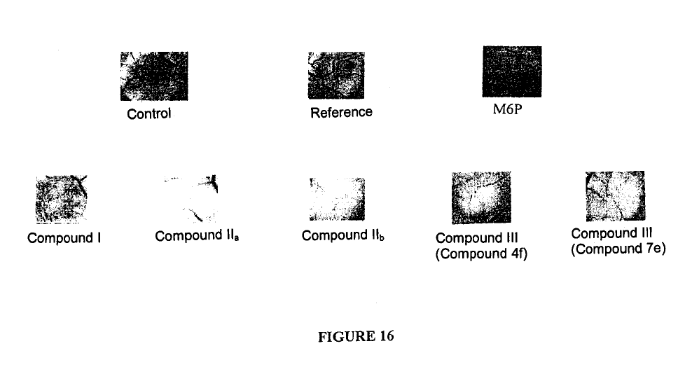

FIG. 16 represents photographs of the vascularization of chick embryos during a conventional test for studying angiogenesis in vivo carried out on the chorioallantoic membrane (CAM).

It should be understood, however, that these examples are only given purely by way of illustration of the invention, of which they in no way constitute any limitation.

### EXAMPLE 1

### Preparation of a Compound of Formula (I)

### 1) SYNTHESIS OF 2-BROMOETHYL-2,3,4,6-TETRA-O-ACETYL-α-D-MANNO-PYRANOSE (Compound 1a)

8 g of acetyl-2,3,4,6-tetra-O-α-D-mannopyranose (20.5 mmol, 1 eq.) dissolved in 70 ml of dichloromethane are reacted with 4.34 ml of 2-bromoethanol (61.5 mmol, 2 eq.) and 15.5 ml of BF₃-Et₂O (123 mmol, 5 eq.). After stirring for 8 hours at ambient temperature, the reaction mixture is diluted in CH₂Cl₂, and washed with water, a saturated solution of NaHCO₃, then again with water. The aqueous phase is then dried over Na₂SO₄, filtered and evaporated. The product is purified by silica gel column chromatography (5/5 v/v ethyl acetate (EtOAc)/petroleum ether (PE)). 2-Bromoethyl-2,3,4,6-tetra-O-acetyl-α-D-mannopyranoside is obtained in the form of a white powder (8.5 g, 91%).

Rf: 0.86 (5/5 v/v EtOAc/PE).

MS (ESI⁺/MeOH): m/z 477.01, 478.95 [M+Na]⁺.

¹H NMR (400.13 MHz, CDCl₃) δ (ppm): 2.00, 2.05, 2.11, 2.16 (4 s, 12H, H_{b}); 3.52 (t, 2H, J₈₋₇=6.0 Hz, H₈); 3.93 (m, 2H, H₇); 4.13 (m, 2H, H₅ and H₆ₐ); 4.27 (dd, 1H, J_{6b-5}=5.8 Hz, J_{6b-6a}=12.6 Hz, H_{6b}); 4.88 (d, 1H, J₁₋₂=1.6 Hz, H₁); 5.27 (dd, 1H, J₂₋₁=2.0 Hz, J₂₋₃=3.2 Hz, H₂); 5.29 (t, 1H, J₄₋₅=J₄₋₃=1.6 Hz, H₄); 5.35 (dd, 1H, J₃₋₂=3.6 Hz, J₃₋₄=10.0 Hz, H₃).

¹³C NMR (100.62 MHz, CDCl₃) δ (ppm): 20.67, 20.70, 20.75, 20.87 (4C_{b}); 29.60 (C₈); 62.41 (C₆); 66.00 (C₄); 68.48 (C₇); 68.93 (C₅); 69.02 (C₃); 69.42 (C₂); 97.75 (C₁); 169.76, 169.86, 170.03, 170.62 (4 Cₐ).

### 2) SYNTHESIS OF 2′-AZIDOETHYL-2,3,4,6-TETRA-O-ACETYL-α-D-MANNO-PYRANOSE (Compound 2a)

5.7 g of 2-bromoethyl-2,3,4,6-tetra-O-acetyl-α-D-mannopyranoside (compound 1) (12.6 mmol, 1 eq.) and 1.64 g of sodium azide (25.16 mmol, 2 eq.) are dissolved in 50 ml of dimethylformamide (DMF). After stirring for 4 hours at a temperature of 65° C., the reaction mixture is diluted in 50 ml of EtOAc and extracted with a saturated solution of NaCl, then washed with distilled water in order to remove the DMF. The organic phase is then dried over Na₂SO₄, filtered and concentrated to give a white solid, 2′-azidoethyl-2,3,4,6-tetra-O-acetyl-α-D-mannopyranoside (5.25 g, 96%).

Rf: 0.86 (5/5 v/v EtOAc/PE).

MS (ESI⁺/MeOH) m/z: 440.12 [M+Na]⁺.

¹H NMR (400.13 MHz, CDCl₃) δ (ppm): 2.00, 2.05, 2.11, 2.16 (4 s, 12H, H_{b}); 3.47 (m, 2H, H₈); 3.67 (m, 1H, H₇ₐ); 3.87 (m, 1H, H_{7b}); 4.05 (ddd, 1H, J₅₋₆ₐ=2.4 Hz, J_{5-6b}=5.2 Hz, J₅₋₄=9.7 Hz, H₅); 4.13 (dd, 1H, J₆ₐ₋₅=2.6 Hz, J_{6a-6b}=12.2 Hz, H₆ₐ); 4.29 (dd, 1H, J_{6b-5}=5.2 Hz, J_{6b-6a}=12.4 Hz, H_{6b}); 4.87 (d, 1H, J₁₋₂=1.6 Hz, H₁); 5.30 (t, 1H, J₄₋₃=J₄₋₅=10.0 Hz, H₄); 5.28 (dd, 1H, J₂₋₁=2.0 Hz, J₂₋₃=3.2 Hz, H₂); 5.36 (dd, 1H, J₃₋₂=3.2 Hz, J₃₋₄=10.0 Hz, H₃).

¹³C NMR (100.62 MHz, CDCl₃) δ (ppm): 20.63, 20.68, 20.71, 20.84 (4C, C_{b}); 50.32 (C₈); 62.42 (C₆); 65.96 (C₄); 67.02 (C₇); 68.82 (C₅ and C₃); 69.36 (C₂); 97.71 (C₁); 169.73, 169.78, 169.98, 170.59 (4C, Cₐ).

### 3) SYNTHESIS OF 2′-AZIDOETHYL-α-D-MANNOPYRANOSE (Compound 3a)

6.8 g of 2′-azidoethyl-2,3,4,6-tetra-O-acetyl-α-D-mannopyranoside (16.3 mmol, 1 eq.) and 880 mg of sodium methanolate (16.3 mmol, 1 eq.) are dissolved in 60 ml of methanol. After stirring for 30 minutes at ambient temperature, the solution is neutralized with Amberlyst IRC-50-H⁺ resin beads, filtered and concentrated. The oil obtained is then purified by silica gel column chromatography with an elution gradient (9/1 v/v CH₂Cl₂/MeOH to 6/4 v/v CH₂Cl₂/MeOH) to give white crystals (2.44 g, 65%).

Rf: 0.4 (8/2 v/v CH₂Cl₂/MeOH).

MS (ESI⁺/MeOH) m/z: 272.11 [M+Na]⁺; 288.02 [M+K]⁺; 521.19 [2M+Na]⁺.

¹H NMR (400.13 MHz, CD₃OD) δ (ppm): 3.41 (t, 2H, J₈₋₇=5.0 Hz, C₈); 3.60 (m, 3H, H₃, H₅ and H₇ₐ); 3.71 (m, 2H, H₄ and H₆ₐ); 3.85 (m, 2H, H₂ and H_{6b}); 3.92 (m, 1H, H_{7b}); 4.81 (d, 1H, J₁₋₂=1.2 Hz, H₁).

¹³C NMR (100.62 MHz, CD₃OD) δ (ppm): 51.76 (C₈); 62.94 (C₆); 67.74 (C₇); 72.08 (C₂); 72.49 (C₄); 68.54, 74.93 (C₃ and C₅); 101.82 (C₁).

### 4) SYNTHESIS OF 2′-AZIDOETHYL-2,3-O—ISOPROPYLIDENE-α-D-MANNO-PYRANOSE (Compound 4a)

Formation of Di-O-Isopropylidene:

9.5 g of 2′-azidoethyl-α-D-mannopyranose (compound 3a) (38.10 mmol, 1 eq.) are suspended in 40 ml of acetone, 23.5 ml of 2,2-dimethoxypropane (190 mmol, 5 eq.) are subsequently added, and then 362 mg of para-toluenesulfonic acid (PTSA) (1.9 mmol, 0.05 eq.) are also added. The mixture is left to stir magnetically at ambient temperature for 4 hours. The reaction is monitored by TLC (6/4 EtOAc/petroleum ether) which then indicates that there is no longer any starting product (Rf=0), a few traces of monoisopropylidene (Rf=0.5) are observed, the product predominantly present being diisopropylidene (Rf=0.8). The PTSA is then neutralized with a 5% NaHCO₃ solution, and the solution is then concentrated in order to remove any trace of acetone. The di-O-isopropylidene obtained is extracted with petroleum ether, and then dried over Na₂SO₄, filtered and concentrated. The product obtained in the form of a yellow oil is pure enough to be reused directly in reaction. The aqueous phase containing the monoisopropylidene is then lyophilized.

Selective Opening:

7.5 g of 2′-azidoethyl-2,3,4,6-di-O-isopropylidene-α-D-mannopyranose (22.9 mmol, 1 eq.) are dissolved in 60 ml of an 80/20 v/v acetic acid/water mixture. After stirring for 2 hours at a temperature of 35° C., the starting product (2-azidoethyl-α-D-mannopyranose) reappears. The solvents are then evaporated off and then co-evaporated off with toluene. The transparent oil obtained is purified by silica gel column chromatography (6/4 v/v EtOAc/PE) to give a slightly yellow oil (5.65 g, 85%).

### Characterization of Diisopropylidene:

Rf: 0.63 (5/5 v/v EtOAc/PE).

MS (ESI⁺/MeOH) m/z: 352.20 [M+Na]⁺; 368.02 [M+K]⁺.

¹H NMR (400.13 MHz, acetone-d₆) δ (ppm): 1.31, 1.32 (2 s, 6H, H_{b} and H_{d}); 1.47, 1.48 (2 s, 6H, H_{c} and Hₑ); 3.50 (t, 2H, J=4.8 Hz, H₈); 3.53 (m, 1H, H₅); 3.72 (m, 3H, H₆ₐ, H₄ and H₇ₐ); 3.82 (dd, 1H, J_{6b-5}=5.8 Hz, J_{6b-6a}=10.6 Hz, H_{6b}); 3.93 (qt, 1H, J=5.2 Hz, H_{7b}); 4.03 (dd, 1H, J₃₋₂=5.6 Hz, J₃₋₄=8.0 Hz, H₃); 4.18 (d, 1H, J₂₋₃=J₂₋₁=5.6 Hz, H₂); 5.09 (s, 1H, H₁).

¹³C NMR (100.62 MHz, acetone-d₆) δ (ppm): 20.11, 29.38 (C_{b} and C_{d}); 27.45, 30.50 (C_{c} and Cₑ); 52.18 (C₈); 63.48, 63.53 (C₅ and C₆); 68.17 (C₇); 74.47 (C₄); 76.78 (C₃); 77.83 (C₂); 99.68 (C₁); 109.76 (Cₐ).

### Characterization of Monoisopropylidene.

Rf: 0.26 (6/4 v/v EtOAc/PE).

MS (ESI⁺/MeOH) m/z: 312.12 [M+Na]⁺; 328.15 [M+K]⁺,

(ESI⁻/MeOH) m/z: 324.12 [M+Cl]⁻.

¹H NMR (400.13 MHz, acetone-d₆+D₂O) δ (ppm): 1.27, 1.41 (2 s, 6H, H_{b} and H_{c}); 3.45 (t, 2H, J=5.0 Hz, H₈); 3.52 (m, 2H, H₄ and H₅); 3.62 (dd, 1H, J₆ₐ₋₅=5.2 Hz, J_{6a-6b}=11.6 Hz, H₆ₐ); 3.67 (m, 1H, H₇ₐ); 3.80 (m, 1H, H_{6b}); 3.93 (m, 1H, H_{7b}); 4.02 (m, 1H, H₃); 4.09 (d, 1H, J₂₋₃=J₂₋₁=5.6 Hz, H₂); 5.03 (s, 1H, H₁).

¹³C NMR (100.62 MHz, acetone-d₆+D₂O) δ (ppm): 2.34, 29.11 (C_{b} and C_{c}); 51.95 (C₈); 62.97 (C₆); 67.74 (C₇); 70.41, 72.62 (C₄ and C₅); 77.30 (C₂); 80.42 (C₃); 98.60 (C₁); 110.60 (Cₐ).

### 5) SYNTHESIS OF 2′-AZIDOETHYL-2,3-O—ISOPROPYLIDENE-4,6-O-(CYCLO-SULFATE)-α-D-MANNOPYRANOSE (Compound 5a)

Formation of the Sulfite:

100 mg of 2′-azidoethyl-2,3-O-isopropylidene-α-D-mannopyranose (compound 4a) (0.345 mmol, 1 eq.) and 169 μl of triethylamine (0.001 mmol, 3 eq.) are dissolved in 2 ml of CH₂Cl₂. The round-bottomed flask is placed in an ice bath and 27 μl of thionyl chloride (0.38 mmol, 1.1 eq.) are slowly added. A white precipitate of triethylammonium chloride appears rapidly and the reaction mixture gradually becomes yellow, and then brown. After stirring for 5 minutes at a temperature of 0° C., there is no longer any starting product, and the desired sulfite is obtained in the form of 2 diastereoisomers (Rf=0.53 and 0.62 (5/5 EtOAc/PE)). The mixture is filtered, and the organic phase is washed with distilled water, a solution of hydrochloric acid (HCl) at 1N, and then again with water. The organic phase is then dried over Na₂SO₄, filtered and concentrated to give a brown solid which is reused directly in reaction.

Formation of the Sulfate:

The crude sulfite (0.345 mmol, 1 eq.) is dissolved in 2 ml of a mixture of CH₂Cl₂/CH₃CN (1/1 v/v). 81 mg of sodium metaperiodate (0.38 mmol, 1.1 eq.), 0.5 ml of water and a grain of ruthenium chloride (1.38×10⁻³ mmol, 0.004 eq.) are successively added. The reaction is exothermic, and an NalO₃ precipitate forms very rapidly. After stirring for 1 hour at ambient temperature, the sulfite has been used up, and the reaction mixture is filtered and diluted in 20 ml of CH₂Cl₂. The organic phase is washed with a 5% NaHCO₃ solution and distilled water, and then dried, filtered and concentrated. The brown solid obtained is dissolved in a minimum amount of CH₂Cl₂ and filtered on silica. The silica is rinsed several times with CH₂Cl₂. A white solid is then obtained (80 mg, 66%).

Rf: 0.58 (5/5 v/v EtOAc/PE).

MS (ESI⁺/MeOH) m/z: 374.13 [M+Na]⁺,

(ESI⁺/MeOH) m/z: 386.08 [M+Cl]⁻.

¹H NMR (400.13 MHz, acetone-d₆) δ (ppm): 1.37, 1.52 (2 s, 6H, C_{b} and C_{c}); 3.55 (m, 2H, H₈); 3.80 (m, 1H, H₇ₐ); 4.29 (m, 1H, H_{7b}); 4.26 (td, 1H, J₅₋₄=J₅₋₆ₐ=10.7 Hz, J_{5-6b}=5.5 Hz, H₅); 4.36 (d, 1H, J₂₋₁=J₂₋₃=6.0 Hz, H₂); 4.43 (dd, 1H, J₃₋₂=5.6 Hz, J₃₋₄=8.0 Hz, H₃); 4.6 (dd, 1H, J₄₋₃=7.6 Hz, J₄₋₅=10.8 Hz, H₄); 4.63 (t, 1H, J₆ₐ₋₅=J_{6a-6b}=10.8 Hz, H₆ₐ); 4.84 (dd, 1H, J_{6b-5}=5.6 Hz, J_{6b-6a}=10.4 Hz, H_{6b}); 5.28 (s, 1H, H₁).

¹³C NMR (100.62 MHz, acetone-d₆) δ (ppm): 27.16, 29.13 (C_{b} and C_{c}); 52.06 (C₈); 60.35 (C₅); 68.75 (C₇); 74.34 (C₆); 74.95 (C₃); 77.88 (C₂); 86.65 (C₄); 99.70 (C₁); 112.07 (Cₐ).

The syntheses of compounds 1a to 5a described above are summarized in the appended FIG. 1.

### 6) SYNTHESIS OF PENT-1-EN-5-YLHEXA(ETHYLENE GLYCOL) (Compound 6a)

25 g of hexa(ethylene glycol) (88.5 mmol, 4.12 eq.) are dissolved in a 50% NaOH solution. After stirring for 30 minutes at a temperature of 100° C., 2.58 ml of 5-bromopent-1-ene (21.85 mmol, 1 eq.) are added. The mixture is kept stirring at a temperature of 100° C. for 15 minutes. The mixture is then diluted in CH₂Cl₂ and the product is extracted with petroleum ether.

Water is added to the CH₂Cl₂ phase, which is reextracted several times with petroleum ether. The organic phases are combined, washed with a minimum amount of water, dried over Na₂SO₄, filtered and concentrated. The yellow oil obtained is purified on a silica gel column (9/1 v/v EtOAc/PE then 9/1 v/v EtOAc/MeOH) to give a yellow liquid oil (3.19 g, 99%).

Rf: 0.14 (5/5 v/v EtOAc/PE).

MS (ESI⁺/MeOH) m/z: 373.27 [M+Na]⁺; 389.20 [M+K]⁺.

¹H NMR (400.13 MHz, CDCl₃) δ (ppm): 1.68 (m, 2H, H₁₄); 2.09 (m, 2H, H₁₅); 3.46 (t, 2H, J₁₃₋₁₄=6.6 Hz, H₁₃); 3.56-3.73 (m, 24H, H₁₋₁₂); 4.99 (m, 2H, H₁₇); 5.81 (m, 2H, H₁₆).

¹³C NMR (100.62 MHz, CDCl₃) δ (ppm): 28.66 (C₁₄); 30.12 (C₁₅); 61.51, 61.58, 69.98, 70.11, 70.21, 70.35, 70.47, 70.58, 72.46 and 72.58 (13C, C₁₋₁₃); 114.59 (C₁₇); 138.18 (C₁₆).

### 7) SYNTHESIS OF (1-THIOACETYLPENT-5-YL)HEXA(ETHYLENE GLYCOL) (Compound 7a)

3.1 g of pent-1-en-5-ylhexa(ethylene glycol) (8.85 mmol, 1 eq.), 3.1 ml of thioacetic acid (44.3 mmol, 5 eq.) and a spatula of azobisisobutyronitrile (AIBN) (100 mg) are dissolved in 12 ml of tetrahydrofuran freshly distilled over sodium. After stirring for 1 hour at reflux (90-100° C.), the mixture is diluted in EtOAc and then washed with a saturated solution of NaHCO₃. The organic phase is dried over Na₂SO₄, filtered and concentrated to give a liquid yellow oil which is purified on a silica gel column (9/1 v/v EtOAc/PE then 9/1 v/v EtOAc/MeOH). A yellow liquid oil is obtained (2.68 g, 71%).

Rf: 0.27 (9/1 v/v EtOAc/MeOH).

MS (ESI⁺/MeOH) m/z: 449.26 [M+Na]⁺, (ESI⁻/MeOH) m/z: 461.17 [M+Cl]⁻.

¹H NMR (400.13 MHz, CDCl₃) δ (ppm): 1.40 (m, 2H, H₁₅); 1.58 (m, 4H, H₁₄ and H₁₆); 1.83 (s, 1H, OH); 2.32 (s, 3H, H₁₉); 2.86 (t, 2H, J₁₇₋₁₆=7.2 Hz, H₁₇); 3.44 (t, 2H, J₁₃₋₁₄=6.6 Hz, H₁₃); 3.56-3.73 (m, 24H, H₁₋₁₂).

¹³C NMR (100.62 MHz, CDCl₃) δ (ppm): 25.25 (C₁₅); 28.90 (C₁₇); 28.99, 29.24 (C₁₄ and C₁₆); 30.52 (C₁₉); 61.55, 69.96, 70.17, 70.42, 70.98 and 72.43 (13C, C₁₋₁₃); 195.84 (C₁₈).

### 8) SYNTHESIS OF (1-THIOMETHOXYTRITYLPENT-5-YL)HEXA(ETHYLENE GLYCOL) (Compound 8a)

Deprotection of the Acetate:

2.6 g of (1-thioacetylpent-5-yl)hexa(ethylene glycol) (60.9 mmol, 1 eq.) are reacted with 3 ml of concentrated HCl in 65 ml of absolute ethanol. After stirring for 20 hours at a temperature of 60° C., the mixture is neutralized with aqueous ammonia, and then concentrated. The solution obtained is then diluted in EtOAc, and the organic phase is washed with water, dried over Na₂SO₄, filtered and concentrated. The browny-black oil obtained is pure enough to be reused directly in reaction.

Protection with Trityl:

The deprotected thiol is placed in the presence of 2.8 g of methoxytrityl chloride (91.3 mmol, 1.5 eq.) in 60 ml of anhydrous THF. After stirring for 24 hours at ambient temperature, the solution is concentrated and purified by silica gel column chromatography (9/1 v/v EtOAc/MeOH) to give a yellow oil (3.65 g, 91%).

Rf: 0.4 (7/3 v/v EtOAc/MeOH).

MS (ESI⁺/MeOH) m/z: 679.34 [M+Na]⁺.

¹H NMR (400.13 MHz, acetone-d₆) δ (ppm): 1.31 (m, 2H, H₁₅); 1.40 (m, 4H, H₁₄ and H₁₆); 2.17 (t, 2H, J₁₇₋₁₈=7.4 Hz, H₁₇); 2.87 (s, 1H, OH); 3.35 (t, 2H, J₁₃₋₁₄=6.4 Hz, H₁₃); 3.47-3.63 (m, 24H, H₁₋₁₂); 3.79 (s, 3H, H₂₁); 6.86-7.42 (m, 14H, H_{Ar}).

¹³C NMR (100.62 MHz, acetone-d₆) δ (ppm): 27.37, 30.16 (3C, C₁₄, C₁₅ and C₁₆), 33.54 (C₁₇); 56.50 (C₂₁); 62.94, 62.94, 67.64, 71.82, 72.19 and 72.33 (13C, C₁₋₁₃); 74.48 (C₁₈); 114.86, 128.32, 129.61, 131.26 and 132.54 (14C, CH_{Ar}); 138.81, 147.37 (3C, C₁₉); 160.12 (C₂₀).

### 9) SYNTHESIS OF O-(1-THIOMETHOXYTRITYLPENT-5-YL)-O-PROPARGYL-HEXA(ETHYLENE GLYCOL) (Compound 9a)

100 mg of (1-thiomethoxytritylpent-5-yl)hexa(ethylene glycol) (0.152 mmol, 1 eq.) are dissolved in 3 ml of freshly distilled THF. 7.3 mg of sodium hydride (0.183 mmol, 1.2 eq.) then 16 μl of 2-bromopropyne (0.213 mmol, 1.4 eq.) are added to the mixture at a temperature of 0° C.

After stirring for 18 hours at ambient temperature, the mixture is concentrated and then purified on a silica gel column (8/2 v/v EtOAc/PE). O-(1-Thiomethoxytritylpent-5-yl)-O-propargyl-hexa(ethylene glycol) is obtained in the form of a whitish oil (103 mg, 97%).

Rf: 0.34 (EtOAc).

MS (ESI⁺/MeOH) m/z: 717.39 [M+Na]⁺.

¹H NMR (400.13 MHz, CDCl₃) δ (ppm): 1.28 (m, 2H, H₁₅); 1.42 (m, 4H, H₁₄ and H₁₆); 2.14 (t, 2H, J₁₇₋₁₆=7.4 Hz, H₁₇); 2.43 (t, 1H, J=2.4 Hz, H_{2′}); 3.36 (t, 2H, J₁₃₋₁₄=6.8 Hz, H₁₃); 3.52-3.71 (m, 24H, H₁₋₁₂); 3.79 (s, 3H, H₂₁); 4.20 (d, 2H, J=2.4 Hz, H_{1′}); 6.79-7.40 (m, 14H, H_{Ar}).

¹³C NMR (100.62 MHz, CDCl₃) δ (ppm): 25.59 (C₁₅); 28.46, 29.19 (C₁₄ and C₁₆); 31.96 (C₁₇); 55.20 (C₂₁); 58.39 (C_{1′}); 65.85 (C_{2′}); 69.10, 70.05, 70.40, 70.56 and 71.17 (13C, C₁₋₁₃); 74.51 (C₁₈); 113.03, 126.44, 127.77, 129.47 and 130.73 (14C, CH_{Ar}); 137.12, 145.32 (3C, C₁₉); 157.94 (C₂₀).

The syntheses of compounds 6a to 9a described above are represented in the appended FIG. 2.

“Click Chemistry” of Compounds 5a and 9a—Functionalization of Mannopyranoside in Position 6 with a Carboxy Group (cf. FIG. 3):

### 10) SYNTHESIS OF {1-[2,3-O—ISOPROPYLIDENE-4,6-O-CYCLOSULFATE-α-D-MANNOPYRANOSYL]ETHYL-1H-1,2,3-TRIAZOL-4-YL}METHYL-[O-(1-THIO-METHOXYTRITYLPENT-5-YL)-O-HEXA(ETHYLENE GLYCOL)] (Compound 10a)

40 mg of 2′-azidoethyl-2,3-O-isopropylidene-4,6-O-cyclosulfate-α-D-mannopyranose (0.11 mmol, 1 eq.) and 88 mg of O-(1-thiomethoxytritylpent-5-yl)-O-propargylhexa(ethylene glycol) (0.13 mmol, 1.1 eq.) are dissolved in 4 ml of a CH₂Cl₂/H₂O mixture (1/1 v/v). 7 mg of CuSO₄, 5H₂O (0.03 mmol, 0.25 eq.) and 11.3 mg of sodium ascorbate (0.06 mmol, 0.5 eq.) are added. After stirring for 24 hours at ambient temperature, the reaction mixture is diluted in CH₂Cl₂, then washed with water. The organic phase is then dried, filtered, concentrated and purified by silica gel column chromatography with an eluent gradient (CH₂Cl₂/MeOH 99/1 v/v to 98/2 v/v) to give a colorless oil (80 mg, 64%).

Rf: 0.4 (9/1 v/v CH₂Cl₂/MeOH).

MS (ESI⁺/MeOH) m/z: 1068.62 [M+Na]⁺,

(ESI⁺/MeOH) m/z: 1080.77 [M+Cl]⁻.

¹H NMR (400.13 MHz, CD₃OD) δ (ppm): 1.36 (m, 6H, H₂₅, H₂₆ and H₂₇); 1.34, 1.49 (2 s, 6H, H_{b} and H_{C}); 2.14 (t, 2H, J₂₈₋₂₇=7.2 Hz, H₂₈); 3.38 (t, 2H, J₂₄₋₂₅=6.4 Hz, H₂₄); 3.44-3.66 (m, 25H, H₅ and H₁₂₋₂₃); 3.78 (s, 3H, H₃₂); 3.97 (m, 1H, H₇ₐ); 4.15 (m, 1H, H_{7b}); 4.27 (m, 3H, H₂, H₃ and H₆ₐ); 4.50 (m, 2H, H_{6b} and H₄); 4.64 (m, 4H, H₈ and H₁₁); 5.12 (s, 1H, H₁); 6.81-7.39 (m, 14H, H_{Ar}); 8.07 (s, 1H, H₉).

¹³C NMR (100.62 MHz, CD₃OD) δ (ppm): 26.34, 28.20 (C_{b} and C_{c}); 26.71 (C₂₆); 29.62 (C₂₇); 30.23 (C₂₅); 33.03 (C₂₈); 51.19 (C₈); 55.79 (C₃₂); 59.79 (C₅); 65.14 (C₁₁); 67.42 (C₇); 70.95, 71.19, 71.49, 71.58, 72.03 (13C, C₁₂₋₂₄); 73.53 (C₆); 74.46, 77.23 (C₂ and C₃); 85.66 (C₄); 98.96 (C₁); 108.26, 111.66 (C₂₉ and Cₐ); 114.11, 127.66, 128.86, 130.73 and 132.02 (14C, CH_{Ar}); 126.04 (C₉); 138.40, 146.86 (4C_{IV}, C₃₀ and C₁₀); 159.71 (C₃₁).

### 11) SYNTHESIS OF {1-[6-CYANO-6-DEOXY-4-O—(SODIUM SULFATE)-2,3-O-ISOPROPYLIDENE-α-D-MANNOPYRANOSYL]ETHYL-1H-1,2,3-TRIAZOL-4-YL}-METHYL-[O-(1-THIOMETHOXYTRITYLPENT-5-YL)-O-HEXA(ETHYLENE GLYCOL)] (Compound 11a)

160 mg of {1-[2,3-O-isopropylidene-4,6-O-cyclosulfate)-α-D-mannopyranosyl]ethyl-1H-1,2,3-triazol-4-yl}methyl-[O-(1-thiomethoxytritylpent-5-yl)-O-hexa(ethylene glycol)](0.15 mmol, 1 eq.) and 15 mg of sodium cyanide (0.31 mmol, 2 eq.) are dissolved in 1.5 ml of DMF. After stirring for 4 hours at ambient temperature, the reaction mixture is diluted in 10 ml of a 5% NaHCO₃ solution (in order to avoid any possible risk of hydrocyanic acid HCN being given off) and washed with CH₂Cl₂. The product is extracted with water, and then the aqueous phase is lyophilized. The yellow powder obtained is purified by silica gel column chromatography (9/1 v/v CH₂Cl₂/MeOH) to give a colorless oil (106 mg, 65%).

Rf: 0.24 (9/1 v/v CH₂Cl₂/MeOH).

MS (ESI⁺/MeOH) m/z: 1117.77 [M+Na]⁺,

(ESI⁺/MeOH) m/z: 1071.63 [M−Na]⁻.

¹H NMR (400.13 MHz, CD₃OD) δ (ppm): 1.31 and 1.50 (2 s, 6H, H_{b} and H_{c}); 1.38 (m, 6H, H₂₆, H₂₇ and H₂₈); 2.15 (t, 2H, J₂₉₋₂₈=7.2 Hz, H₂₉); 2.70 (dd, 1H, J₆ₐ₋₅=8.8 Hz, J_{6a-6b}=17.2 Hz, H₆ₐ); 3.02 (dd, 1H, J_{6b-5}=3.0 Hz, J_{6b-6a}=17.0 Hz, H_{6b}); 3.38 (t, 2H, J₂₅₋₂₆=6.4 Hz, H₂₅); 3.48-3.66 (m, 26H, H_{4.5} and H₁₃₋₂₄); 3.78 (s, 3H, H₃₃); 3.93 (m, 1H, H₈ₐ); 4.10 (d, 1H, J₂₋₃=J₂₋₁=4.8 Hz, H₂); 4.19 (m, 2H, H₈ b and H₃); 4.65 (m, 4H, H₉ and H₁₂); 5.00 (s, 1H, H₁); 6.82-7.39 (m, 14H, H_{Ar}); 8.04 (s, 1H, H₁₀).

¹³C NMR (100.62 MHz, CD₃OD) δ (ppm): 21.83 (C₆); 26.60, 28.05 (C_{b} and C_{c}); 26.69 (C₂₇); 29.62 (C₂₈); 30.17 (C₂₆); 33.04 (C₂₉); 51.17 (C₉); 55.81 (C₃₃); 64.87 (C₁₂); 66.66, 76.70 (C₄ and C₅); 67.02 (C₈); 70.47, 70.99, 71.20, 71.26, 71.35 and 72.02 (13C, C₁₃₋₂₅); 77.54 (C₂); 77.92 (C₃); 98.52 (C₁); 110.98 (C₃₀ and Cₐ); 118.96 (C₇); 114.12, 127.67, 128.87, 130.72 and 132.01 (14C, CH_{Ar}); 125.87 (C₁₀); 138.38, 146.00 and 146.84 (4C_{IV}, C₃₁ and C₁₁); 159.71 (C₃₂).

### 12) SYNTHESIS OF {1-[(6,7-DIDEOXY-4-O-(SODIUM SULFATE)-2,3-O-ISO-PROPYLIDENE-α-D-MANNOHEPTOPYRANOSYL)URONIC ACID]ETHYL-1H-1,2,3-TRIAZOL-4-YL}METHYL-[O-(1-PENT-5-YL)-O-HEXA(ETHYLENE GLYCOL)](Compound 12a)

200 mg of {1-[6-cyano-6-deoxy-4-O-(sodium sulfate)-2,3-O-isopropylidene-α-D-manno-pyranosyl]ethyl-1H-1,2,3-triazol-4-yl}methyl-[O-(1-thiomethoxytritylpent-5-yl)-O-hexa(ethylene glycol)](0.18 mmol, 1 eq.) and 60 mg of sodium hydroxide (NaOH) (1.46 mmol, 8 eq.) are dissolved in 1.5 ml of a 30% aqueous hydrogen peroxide solution. The solution is stirred at ambient temperature. At 12 hours and 24 hours of reaction, 60 mg of NaOH and 1.5 ml of H₂O₂ are added to the reaction medium. After 48 hours, the solution is neutralized with Amberlyst H⁺ resins, before being filtered and lyophilized. The product obtained is then purified by silica gel column chromatography with an elution gradient (9/1 v/v CH₂Cl₂/MeOH to 5/5 v/v NH₄OH/iPrOH) to give a yellow oil (80 mg, 52%).

Rf: 0 (8/2 v/v CH₂Cl₂/MeOH).

MS (ESI⁺/MeOH) m/z: 864.34 [M+Na]⁺.

¹H NMR (400.13 MHz, D₂O) δ (ppm): 1.35 and 1.52 (2 s, 6H, H_{b} and H_{c}); 1.44 (m, 2H, H₂₇); 1.60 (m, 2H, H₂₆); 1.73 (m, 2H, H₂₈); 2.29 (dd, 1H, J₆ₐ₋₅=10.6 Hz, J_{6a-6b}=15.0 Hz, H₆ₐ); 2.80 (dd, 1H, J_{6b-5}=2.0 Hz, J_{6b-6a}=15.2 Hz, H_{6b}); 2.89 (t, 2H, J₂₉₋₂₈=8.0 Hz, H₂₉); 3.53 (t, 2H, J₂₅₋₂₆=6.6 Hz, H₂₅); 3.50-3.69 (m, 26H, H₅ and H₁₃₋₂₄); 3.88 (m, 1H, H₈ₐ); 4.17 (m, 2H, H₈ b and H₄); 4.19 (d, 1H, J₂₋₃=J₂₋₁=5.6 Hz, H₂); 4.30 (m, 1H, H₃); 4.68 (m, 4H, H₉ and H₁₂); 4.95 (s, 1H, H₁); 8.12 (s, 1H, H₁₀).

¹³C NMR (100.62 MHz, D₂O) δ (ppm): 23.80 (C₂₈); 24.24 (C₂₇); 25.45 and 26.80 (C_{b} and C_{c}); 28.06 (C₂₆); 38.92 (C₆); 49.99 (C₉); 50.89 (C₂₉); 62.95 (C₁₂); 65.47 (C₈); 66.41 (C₅); 66.54, 68.69, 69.07, 69.40, 69.53 and 70.70 (13C, C₁₃₋₂₅); 75.21 (C₂); 76.00 (C₃); 78.30 (C₄); 95.99 (C₁); 110.39 (Cₐ); 125.55 (C₁₀); 143.85 (C₁₁); 177.75 (C₇).

### 13) SYNTHESIS OF {1-[(6,7-DIDEOXY-α-D-MANNOHEPTOPYRANOSYL)-URONIC ACID]ETHYL-1H-1,2,3-TRIAZOL-4-YL}METHYL-[O-(1-PENT-5-YL)-O-HEXA(ETHYLENE GLYCOL)] (Compound 13a)

60 mg of {1-[(6,7-dideoxy-4-O-(sodium sulfate)-2,3-O-isopropylidene-α-D-manno-heptopyranosyl)uronic acid]ethyl-1H-1,2,3-triazol-4-yl}methyl-[O-(1-pent-5-yl)-O-hexa(ethylene glycol)](0.07 mmol, 1 eq.) are dissolved in 2 ml of a mixture of MeOH/THF (1/1 v/v), then reacted with Amberlyst 15-H⁺ resins for 36 hours. The resins are then filtered off and the solution is neutralized with a 5% NaHCO₃ solution. The organic solvents are evaporated off and the remaining water is lyophilized. The mixture is taken up in methanol and the insoluble NaHCO₃ is filtered off. The oil obtained is pure enough to be reused directly in reaction (40 mg, 80%).

Rf: 0.18 (5/5 v/v EtOAc/MeOH).

MS (ESI⁺/MeOH) m/z: 765.86 [M−3H+3Na]⁺.

¹H NMR (400.13 MHz, CD₃OD) δ (ppm): 1.49 (m, 2H, H₂₇); 1.61 (m, 2H, H₂₆); 1.80 (m, 2H, H₂₈); 2.41 (dd, 1H, J₆ₐ₋₅=10.2 Hz, J_{6a-6b}=16.2 Hz, H₆ₐ); 2.84 (m, 3H, H₂₉ and H_{6b}); 3.49 (t, 2H, J₂₅₋₂₆=6.4 Hz, H₂₅); 3.40-3.79 (m, 28H, H₂₋₅ and H₁₃₋₂₄); 3.92 (m, 1H, H₈ₐ); 4.22 (m, 1H, H_{8b}); 4.71 (d, 1H, J₁₋₂=1.2 Hz, H₁); 4.87 (m, 2H, H₉); 4.92 (m, 2H, H₁₂); 8.65 (s, 1H, H₁₀).

¹³C NMR (100.62 MHz, D₂O) δ (ppm): 23.79 (C₂₈); 24.23 (C₂₇); 28.06 (C₂₆); 36.51 (C₆); 50.67 (C₉); 50.90 (C₂₉); 62.55 (C₁₂); 65.29 (C₈); 67.88, 69.08, 69.54 and 70.70 (13C, C₁₃₋₂₅); 52.32, 69.36, 69.82, 70.16 (4C, C₂₋₅); 99.48 (C₁); 109.39 (C₁₁); 146.74 (C₁₀); 175.27 (C₇).

“Click Chemistry” of Compounds 5a and 9a—Functionalization of Mannopyranoside in Position 6 with an Azido Group (cf. FIG. 4):

### 14) SYNTHESIS OF {1-(6-AZIDO-6-DEOXY-2,3-O-ISOPROPYLIDENE-4-O-(SODIUM SULFATE)-α-D-MANNOPYRANOSYL)ETHYL-1H-1,2,3-TRIAZOL-4-YL}-METHYL-[O-(1-THIOPENT-5-YL)-O-HEXA(ETHYLENE GLYCOL)] (Compound 14a)

530 mg of {1-[2,3-O-isopropylidene-4,6-O-(cyclosulfate)-α-D-mannopyranosyl]ethyl-1H-1,2,3-triazol-4-yl}methyl-[O-(1-thiomethoxytritylpent-5-yl)-O-hexa(ethylene glycol)](0.51 mmol, 1 eq.) and 65 mg of sodium azide (1.00 mmol, 2 eq.) are reacted in 10 ml of DMF. The same protocol as for 2′-azidoethyl-2,3,4,6-tetra-O-acetyl-α-D-mannopyranose (compound 2a) is then followed (Example 1). A yellow oil is then obtained (350 mg, 62%).

Rf: 0.15 (8.5/1 v/v CH₂Cl₂/MeOH).

### 15) SYNTHESIS OF {1-(6-AZIDO-6-DEOXY-2,3-α-D-MANNOPYRANOSYL)-ETHYL-1H-1,2,3-TRIAZOL-4-YL}METHYL-[O-(1-THIOPENT-5-YL)-O-HEXA-(ETHYLENE GLYCOL)] (Compound 15a)

200 mg of {1-(6-azido-6-deoxy-2,3-O-isopropylidene-4-O-(sodium sulfate)-α-D-manno-pyranosyl)ethyl-1H-1,2,3-triazol-4-yl}methyl-[O-(1-thiopent-5-yl)-O-hexa(ethylene glycol)](0.18 mmol, 1 eq.) are reacted with 50 mg of ceric ammonium nitrate (CAN) (0.09 mmol, 0.5 eq.) in 4 ml of a CH₃CN/H₂O mixture (95/5 v/v). After stirring for 4 hours at 60° C., the reaction mixture is diluted in CH₂Cl₂, and washed several times with water, and the aqueous phase is lyophilized. The yellow oil obtained is purified by silica gel column chromatography with an eluent gradient (90/10 v/v CH₂Cl₂/MeOH to 80/20 v/v CH₂Cl₂/MeOH) to give {1-(6-azido-6-deoxy-4-O-sodium sulfate-α-D-mannopyranosyl)ethyl-1H-1,2,3-triazol-4-yl}methyl-[O-(1-thiopent-5-yl)-O-hexa(ethylene glycol)] in the form of a colorless oil (100 mg, 72%).

This compound is then reacted with Amberlyst H⁺ resins in 6 ml of a mixture of MeOH/THF (1/1 v/v) for 24 hours. The same protocol as for {1-[(6,7-dideoxy-α-D-manno-heptopyranosyl)uronic acid]ethyl-1H-1,2,3-triazol-4-yl}methyl-[O-(1-pent-5-yl)-O-hexa(ethylene glycol)](compound 13a) is then followed (Example 1). A yellow oil is then obtained (150 mg, 53%).

Rf: 0.25 (91/v/v CH₂Cl₂/MeOH).

¹H NMR (400.13 MHz, CD₃OD) δ (ppm): 1.47 (m, 2H, H₂₆); 1.60 (m, 2H, H₂₅); 1.71 (m, 2H, H₂₇); 2.70 (t, 2H, J₂₈₋₂₇=7.2 Hz, H₂₈); 3.48 (t, 2H, J₂₄₋₂₅=6.2 Hz, H₂₄); 3.19-3.78 (m, 30H, H₂₋₆ and H₁₂₋₂₃); 3.88 (m, 1H, H₇ₐ); 4.13 (m, 1H, H_{7b}); 4.63 (m, 4H, H₈ and H₁₁); 4.72 (s, 1H, H₁); 8.03 (s, 1H, H₉).

¹³C NMR (100.62 MHz, CD₃OD) δ (ppm): 26.13 (C₂₆); 30.07 (C₂₇); 30.36 (C₂₅); 39.66 (C₂₈); 51.34 (C₈); 62.85 (C₆); 65.05 (C₁₁); 66.79 (C₇); 68.38, 70.81, 71.24, 71.59, 71.93, 72.15, 72.51 and 75.01 (17C, C₂₋₅ and C₁₂₋₂₄); 101.70 (C₁); 132.57 (C₉); 161.04 (C₁₀).

Synthesis of the Compound of Formula (I), in which a is a Gold Nanoparticle:

Two solutions, one containing 60 mg of tetrachloroauric acid (HAuCl₄) (0.18 mmol, 1 eq.) and 250 ml of water, and the other containing 150 mg of sodium citrate (0.5 mmol, 2.78 eq.) dissolved in 10 ml of water, are heated in parallel at a temperature of 60° C. for 10 minutes. The hot sodium citrate solution is then added to the HAuCl₄ solution, and the mixture is brought to a temperature of 120° C. for 2.5 hours. The solution gradually turns from gray to burgundy red, which indicates that the gold nanoparticles have formed. Once the solution has returned to ambient temperature, 50 mg of glycoconjugate solubilized in 1 ml of MeOH are added and left to stir at ambient temperature for 48 hours. The gold nanoparticles are then precipitated by adding a saturated NaCl solution. After standing overnight, the supernatant is removed and the gold nanoparticles are centrifuged for 30 minutes at a speed of 14 000 rpm. They are then washed several times with water, then with methanol, and dried in the open air.

Size of the gold nanoparticles A: 6-7 nm,

Size of the compounds of formula (I): 7-8 nm.

### EXAMPLE 2

### Preparation of a Compound of Formula (II)

### 1) SYNTHESIS OF 3,6,8-DIOXAOCTYL 2,3,4,6-TETRA-O-ACETYL-α-D-MANNO-PYRANOSIDE (Compound 1b)

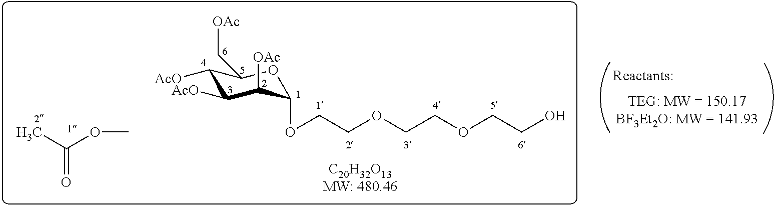

60 ml (0.56 mol-8 eq.) of BF₃Et₂O are added dropwise, under an argon atmosphere, to a solution containing 25.7 g (0.07 mol-1 eq.) of α-D-mannose pentaacetate and 26 ml (0.21 mol-3 eq.) of triethylene glycol in 150 ml of anhydrous dichloromethane cooled to a temperature of 0° C. The reaction mixture is then left to return to ambient temperature. The mixture is kept stirring overnight, and the reaction is monitored by TLC (90/10 v/v Et₂O/MeOH). Once the reaction is complete, the reaction mixture is washed successively with twice 100 ml of a saturated aqueous solution of NaHCO₃, with twice 100 ml of distilled water and, finally, with 100 ml of a brine solution. The organic phase is dried over Na₂SO₄, filtered and evaporated under reduced pressure. The reaction crude is then purified by silica gel column chromatography, elution being carried out with 60/40 v/v PE/Et₂O.

Physical appearance: Yellow oil.

Yield: 79%.

Rf: 0.43 (90/10 v/v Et₂O/MeOH).

MS: (ESI⁺/MeOH) m/z: 503.1 [M+Na]⁺,

(ESI⁻/MeOH) m/z: 479.3 [M−H]⁻.

¹H NMR (400.13 MHz, CDCl₃) δ (ppm): 2.05, 2.09, 2.12, 2.13 (4 s, 12H, H_{2″}); 3.55-3.68 (m, 12H, H_{1′}, H_{2′}, H_{3′}, H_{4′}, H_{5′}, H_{6′}); 4.05-4.11 (m, 1H, H₆ₐ); 4.17-4.24 (m, 2H, H₅ and H_{6b}); 5.18 (d, 1H, ³J_{H1-H2}=1.8 Hz, H₁); 5.20 (dd, 1H, ³J_{H2-H1}=1.9 Hz, ³J_{H2-H3}=3.2 Hz, H₂); 5.25 (t, 1H, ³J_{H4-H3}=³J_{H4-H5}=9.9 Hz, H₄); 5.36 (dd, 1H, ³J_{H3-H2}=3.3 Hz, ³J_{H3-H4}=10.1 Hz, H₃).

¹³C NMR (100.62 MHz, CDCl₃) δ (ppm): 20.5 (2C), 20.6, 20.7 (4C, C_{2″}); 61.03 (1C, C_{6′}); 62.5 (1C, C₆); 66.0 (1C, C₄); 66.53 (1C, C_{1′}); 68.1 (1C, C₅); 68.8 (1C, C₃); 69.94 (2C, C_{3′} and C_{4′}); 70.1 (1C, C₂); 70.44 (1C, C_{2′}); 72.60 (1C, C_{5′}); 91.8 (1C, C₁); 169.8, 170.1, 170.2 and 170.9 (4C, C_{1″}).

### 2) SYNTHESIS OF 3,6,8-DIOXAOCTYL BIS(2,3,4,6-TETRA-O-ACETYL-α-D-MANNOPYRANOSIDE) (Compound 2b)

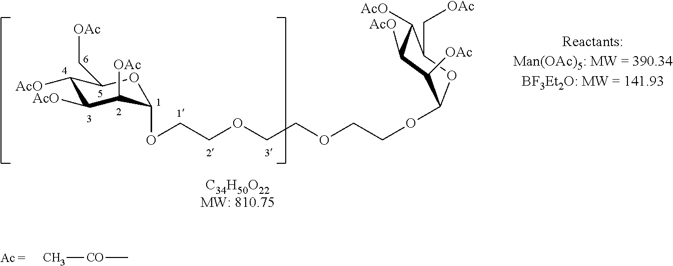

35.5 ml (0.28 mol-10 eq.) of BF₃Et₂O are added dropwise, under an argon atmosphere, to a solution containing 13.4 g (27.9 mmol-1 eq.) of compound 1b and 10 g (27.9 mmol-1 eq.) of α-D-mannose pentaacetate in 120 ml of anhydrous dichloromethane cooled to a temperature of 0° C. The reaction mixture is then left to return to ambient temperature. The mixture is kept stirring overnight, and the reaction is monitored by TLC (90/10 v/v Et₂O/MeOH). Once the reaction is complete, the reaction mixture is washed successively with twice 80 ml of a saturated aqueous solution of NaHCO₃, with twice 80 ml of distilled water and, finally, with 80 ml of a brine solution. The organic phase is dried over Na₂SO₄, filtered and evaporated under reduced pressure. The reaction crude is subsequently purified by silica gel column chromatography, elution being carried out with 60/40 v/v PE/Et₂O.

Physical appearance: White foam.

Yield: 87%.

Rf: 0.61 (Et₂O).

MS: (ESI⁺/MeOH) m/z: 833.4 [M+Na]⁺; 811.2 [M+H]⁺,

(ESI⁻/MeOH) m/z: 809.3 [M−H]⁻; 845.4 [M+Cl]⁻.

¹H NMR (400.13 MHz, CDCl₃) δ (ppm): 1.98, 2.00, 2.11, 2.13 (4 s, 24H, H_{2″}); 3.42-3.46 (m, 6H, H₃, H₅, H_{6b}); 3.51 (dd, 2H, ³J_{H6a-H5}=7.1 Hz, ²J_{H6a-H6b}=2.0 Hz, H₆ₐ); 3.53 (dd, 2H, ³J_{H4-H3}=8.5 Hz, ³J_{H4-H5}=2.7 Hz, H₄); 3.58 (s, 4H, H₃); 3.60 (t, 4H, ³J_{H2′-H1′}=3.4 Hz, H_{2′}); 3.63 (dd, 2H, ³J_{H2-H3}=9.2 Hz, ³J_{H2-H1}=1.8 Hz, H₂); 3.67 (t, 4H, ³J_{H1′-H2′}=3.3 Hz, H_{1′}); 5.40 (dd, 2H, ³J_{H1-H2}=1.5 Hz, H₁).

¹³C NMR (100.62 MHz, CDCl₃) δ (ppm): 20.6, 20.7, 20.75, 20.9 (8C, C_{2″}); 62.26 (2C, C₆); 66.53 (2C, C_{1′}); 69.94 (2C, C_{3′}); 70.44 (2C, C_{2′}); 70.46 (2C, C₄); 73.34 (2C, C₂); 74.80 (2C, C₃); 76.66 (2C, C₅); 103.79 (2C, C₁).

### 3) SYNTHESIS OF 3,6,8-DIOXAOCTYL BIS(α-D-MANNOPYRANOSIDE) (Compound 3b)

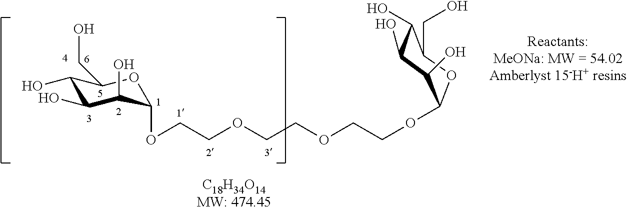

2.20 g (4 mmol-0.3 eq.) of MeONa are added to a solution of 100 ml of methanol containing 11 g (13 mmol-1 eq.) of compound 2b. The reaction is monitored by TLC 60/40 v/v iPrOH/NH₄ OH. After 30 minutes, the reaction medium is neutralized with Amberlyst 15-H⁺ acid resins. Next, the resins are filtered, and rinsed with methanol. The crude obtained after concentration is purified by silica gel column chromatography with an elution gradient (80/20 v/v iPrOH/NH₄ OH to 60/40 v/v iPrOH/NH₄OH).

Physical appearance: Light beige foam.

Yield: 97%.

Rf: 0.35 (60/40 v/v iPrOH/NH₄OH).

MS (ESI⁺/MeOH) m/z: 497.3 [M+Na]⁺; 475.4 [M+H]⁺,

(ESI⁻/MeOH) m/z: 473.3 [M−H]⁻.

¹H NMR (400.13 MHz, MeOD) δ (ppm): 3.41-3.46 (m, 6H, H₃, H₅, H_{6b}); 3.53 (dd, 2H, ³J_{H6a-H5}=7.5 Hz, 2J_{H6a-H6b}=2.1 Hz, H₆ₐ); 3.54 (dd, 2H, ³J_{H4-H3}=8.5 Hz, ³J_{H4-H5}=3.1 Hz, H₄); 3.57 (m, 4H, H_{3′}); 3.60 (t, 4H, ³J_{H2′-H1′}=3.3 Hz, H_{2′}); 3.64 (dd, 2H, ³J_{H2-H3}=8.7 Hz, ³J_{H2-H1}=1.5 Hz, H₂); 3.66 (t, 4H, ³J_{H1′-H2′}=3.3 Hz, H_{1′}); 5.38 (dd, 2H, ³J_{H1-H2}=1.9 Hz, H₁).

¹³C NMR (100.62 MHz, MeOD) δ (ppm): 63.08 (2C, C₆); 66.64 (2C, C₁₁); 70.11 (2C, C_{3′}); 70.44 (2C, C_{2′}); 70.51 (2C, C₄); 73.35 (2C, C₂); 75.20 (2C, C₃); 76.83 (2C, C₅); 104.19 (2C, C₁).

### 4) SYNTHESIS OF 3,6,8-DIOXAOCTYL BIS(6-DEOXY-6-IODO-α-D-MANNO-PYRANOSIDE) (Compound 4b)

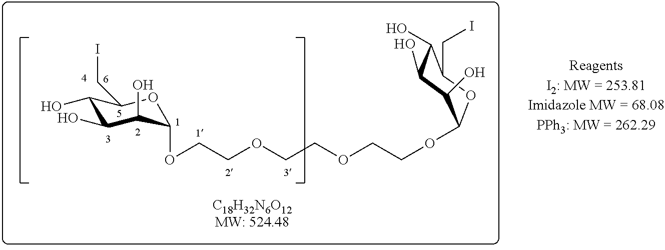

0.4 g (1.58 mmol-1.5 eq.) of iodine, 0.5 g (1.05 mmol-1 eq.) of compound 3b, 0.42 g (1.58 mmol-1.5 eq.) of triphenylphosphine (PPh₃) and 145 mg (2.1 mmol-2 eq.) of imidazole are ground together in a small round-bottomed flask using a glass rod. The reaction mixture is heated at a temperature of 100° C. with stirring for 10 minutes. The iodination reaction is monitored by TLC (90/10 v/v CH₂Cl₂/MeOH). The mixture is then cooled to ambient temperature and then solubilized in methanol. The reaction crude is then concentrated under reduced pressure, and purified by silica gel column chromatography (90/10 v/v CH₂Cl₂/MeOH).

### 5) SYNTHESIS OF 3,6,8-DIOXAOCTYL BIS(6-DEOXY-6-AZIDO-α-D-MANNO-PYRANOSIDE) (Compound 5b)

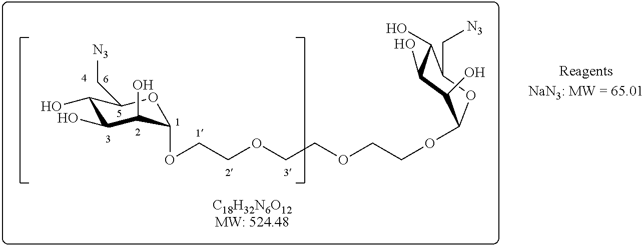

140 mg of compound 4b (0.14 mmol, 1 eq.) and 38 mg of sodium azide (0.57 mmol, 4 eq.) are mixed and ground with a mortar for 5 minutes, and the mixture is then immersed in an oil bath at a temperature of 80° C. After manual stirring for 20 minutes, the reaction mixture is directly purified by silica gel column chromatography (9/1 v/v CH₂Cl₂/MeOH) to give a yellow oil (53 mg, 70%).

The scheme for synthesis of the compound of formula (II) is represented in the appended FIG. 5.

### EXAMPLE 3

### Preparation of a Compound of Formula (III)

### 1) SYNTHESIS OF METHYL 6-MONOMETHOXYTRITYL-α-D-MANNO-PYRANOSIDE (Compound 1e)

10 g of methyl α-D-mannopyranoside (51.5 mmol, 1 eq.) and 1.9 g of DMAP (15.45 mmol, 0.3 eq.) are dissolved in 80 ml of pyridine. 24 g of monomethoxytrityl chloride (77.25 mmol, 1.5 eq.) are added to the mixture in fractions. The reaction is complete after 1.5 hours, and the reaction medium is then diluted in EtOAc and washed successively with a 2N solution of HCl, a 5% solution of NaHCO₃ and water. The organic phase is dried over Na₂SO₄, filtered and concentrated. The product is then purified by flash chromatography on silica gel with an elution gradient (CH₂Cl₂ to 95/5 v/v CH₂Cl₂/MeOH) to give a slightly yellow powder (21.6 g, 90%).

Rf: 0.43 (95/5 v/v CH₂Cl₂/MeOH).

MS (ESI⁺/MeOH) m/z: 489.45 [M+Na]⁺; 955.67 [2M+Na]⁺,

(ESI⁺/MeOH) m/z: 465.28 [M−H]⁻.

¹H NMR (400.13 MHz, CDCl₃) δ (ppm): 1.65-3.00 (3 m, 3H, —OH); 3.30 (s, 3H, —OCH₃); 3.32 (m, 1H, H₆ₐ); 3.37 (m, 1H, H_{6b}); 3.55-3.80 (m, 4H, H₂, H₃, H₄ and H₅); 3.72 (s, 3H, H₁₃); 4.65 (d, 1H, J₁₋₂=1.4 Hz, H₁); 6.70-7.40 (m, 14H, H_{9,10,11}).

¹³C NMR (100.62 MHz, CDCl₃) δ (ppm): 54.90 (—OCH₃); 55.21 (C₁₃); 64.98 (C₆); 69.91, 69.99, 70.23 and 71.6 (C₂, C₃, C₄ and C₅); 87.15 (C₇); 100.64 (C₁); 113.38, 127.19, 127.85, 128.42, 130.42 (14C, C_{9,10,11}); 135.32, 144.17, 144.26 (C₈); 158.72 (C₁₂).

### 2) SYNTHESIS OF METHYL 2,3,4-TRI-O-BENZYL-6-MONOMETHOXYTRITYL-α-D-MANNOPYRANOSIDE (Compound 2e)

5 g of methyl 6-monomethoxytrityl-α-D-mannopyranoside (10.72 mmol, 1 eq.) are dissolved in 19 ml of benzyl bromide (160.8 mol, 15 eq.). 15 g of KOH (268 mmol, 20 eq.) are then added and the mixture is heated to a temperature of 80° C. After 1 hour of reaction, the reaction medium is diluted in CH₂Cl₂, and then the organic phase is washed with distilled water, dried over Na₂SO₄, filtered and concentrated. The product is then purified by silica gel chromatography with an elution gradient (PE to 5/5 v/v PE/Et₂O) to give an ecru foam (7.1 g, 90%).

Rf: 0.62 (6/4 v/v PE/Et₂O).

MS (ESI⁺/MeOH) m/z: 759.45 [M+Na]⁺.

¹H NMR (400.13 MHz, CDCl₃) δ (ppm): 3.19 (dd, 1H, J₆ₐ₋₅=5.2 Hz, J_{6b-6a}=9.8 Hz, H₆ₐ); 3.28 (s, 3H, —OCH₃); 3.43 (dd, 1H, H_{6b-5}=1.7 Hz, J_{6b-6a}=9.8 Hz, H_{6b}); 3.64 (s, 3H, H₁₃); 3.69 (m, 1H, H₅); 3.73 (dd, 1H, J₂₋₁=1.8 Hz, J₂₋₃=3.1 Hz, H₂); 3.79 (dd, 1H, J₃₋₂=3.2 Hz, J₃₋₄=9.3 Hz, H₃); 3.95 (t, 1H, J₄₋₃=J₄₋₅=9.6 Hz, H₄); 4.42-4.69 (m, 4H, Hₐ); 4.74 (s, 1H, H₁); 6.66-7.49 (m, 29H, H_{9,10,11,c,d,e}).

¹³C NMR (100.62 MHz, CDCl₃) δ (ppm): 55.00 (—OCH₃); 55.22 (C₁₃); 63.85 (C₆); 71.91 (C₅); 75.32 (C₄); 75.59 (C₂); 80.37 (C₃); 72.43, 72.86 and 75.20 (Cₐ); 86.01 (C₇); 98.87 (C₁); 113.18, 128.61, 127.45-127.90, 128.21-128.74, 130.64 (29C, C_{9,10,11,c,d,e}); 135.92, 138.48, 138.76, 138.81, 144.72 and 144.90 (6C, C_{b} and C₈); 158.52 (C₁₂).

### 3) SYNTHESIS OF METHYL 2,3,4-TRI-O-BENZYL-α-D-MANNOPYRANOSIDE (Compound 3e)

7.12 g of methyl 2,3,4-tri-O-benzyl-6-monomethoxytrityl-α-D-mannopyranoside (9.66 mmol, 1 eq.) are dissolved in 80 ml of a 95/5 v/v mixture of CH₃CN/H₂O. 530 mg of CAN (0.97 mmol, 0.1 eq.) are then added and the mixture is heated at a temperature of 60° C. for minutes. The reaction medium is then diluted with CH₂Cl₂, and then the organic phase is washed with distilled water, dried over Na₂SO₄, filtered and concentrated. The product is then purified by flash chromatography on silica gel with an elution gradient (3/7 v/v PE/Et₂O) to give a colorless oil (4.26 g, 95%).

Rf: 0.5 (4/6 v/v Et₂O/PE).

MS (ESI⁺/MeOH) m/z: 487.12 [M+Na]⁺; 951.34 [2M+Na]⁺.

¹H NMR (400.13 MHz, CDCl₃) δ (ppm): 1.88 (s, 1H, —OH); 3.25 (s, 3H, —OCH₃); 3.57 (m, 1H, H₅); 3.73 (m, 3H, H₆ₐ and H₂); 3.80 (dd, 1H, J₆ₐ₋₅=3.0 Hz, J_{6a-6b}=11.8 Hz, H_{6b}); 3.85 (dd, 1H, J₃₋₂=3.0 Hz, J₃₋₄=9.4 Hz, H₃); 3.92 (t, 1H, J₄₋₃=J₄₋₅=9.6 Hz, H₄); 4.61 (m, 2H, Hₐ); 4.65 (m, 3H, H₁ and Hₐ); 4.73 (d, 2H, J=12.4 Hz, Hₐ); 4.89 (d, 2H, J=10.8 Hz, Hₐ); 7.21-7.305 (m, 15H, H_{c,d,e}).

¹³C NMR (100.62 MHz, CDCl₃) δ (ppm): 54.73 (—OCH₃); 62.37 (C₆); 71.99 (C₅); 74.64 (C₂); 74.83 (C₄); 72.17, 72.90, 75.16 (3Cₐ); 80.17 (C₃); 99.29 (C₁); 127.54-128.35 (C_{c,d,e}); 138.21, 138.37, 138.42 (C_{b}).

### 4) SYNTHESIS OF METHYL 2,3,4-TRI-O-BENZYL-6-DEOXY-6-OXY-α-D-MANNOPYRANOSIDE (Compound 4e)

500 mg of methyl 2,3,4-tri-O-benzyl-α-D-mannopyranoside (1.1 mmol, 1 eq.) are dissolved in 12 ml of CH₂Cl₂, before adding 2.5 g of 4 Å molecular sieve and 464 mg of pyridinium chlorochromate (2.15 mmol, 2 eq.). After stirring for 1 hour at ambient temperature, the reaction medium is filtered on celite then on active carbon, before being purified by chromatography on silica (3/7 v/v EtOAc/PE) to give a transparent oil (250 mg, 50%).

Rf: 0.2 (EtOAc/PE 5/5 v/v).

MS (ESI⁺/MeOH) m/z: 485.46 [M+Na]⁺.

¹H NMR (400.13 MHz, CDCl₃) δ (ppm): 3.31 (s, 3H, —OCH₃); 3.70 (t, 1H, J₂₋₁=J₂₋₃=2.8 Hz, H₂); 3.88 (dd, 1H, J₃₋₂=3.0 Hz, J₃₋₄=7.8 Hz, H₃); 3.98 (t, 1H, J₄₋₅=J₄₋₃=8.2 Hz, H₄); 4.01 (m, 1H, H₅); 4.54-4.76 (m, 6H, Hₐ); 4.78 (d, 1H, J₁₋₂=2.8 Hz, H₁); 7.19-7.29 (m, 15H, H_{c,d,e}); 9.66 (s, 1H, H₆).

¹³C NMR (100.62 MHz, CDCl₃) δ (ppm): 55.51 (—OCH₃); 72.27, 72.91 and 74.66 (3Cₐ); 74.17 (C₂); 74.36 (C₄); 76.00 (C₅); 79.15 (C₃); 99.42 (C₁); 127.61-128.41 (C_{c,d,e}); 137.73, 138.02 (3C_{b}); 197.86 (C₆).

### 5) SYNTHESIS OF METHYL 2,3,4-TRI-O-BENZYL-6-DEOXY-6-ALLYL-α-D-MANNOPYRANOSIDE (Compound 5e)

693 mg of methyltriphosphonium bromide (1.95 mmol, 1.2 eq.) are dissolved in 12 ml of anhydrous THF, before adding, under an argon atmosphere at a temperature of −5° C., 2 ml of BuLi (4.88 mmol, 3 eq.). Stirring is maintained for 30 minutes at a temperature of −5° C. The solution turns yellow and then 751 mg of methyl 2,3,4-tri-O-benzyl-6-deoxy-6-oxy-α-D-mannopyranoside (1.63 mmol, 1 eq.), dissolved beforehand in 8 ml of anhydrous THF, are added to the solution at a temperature of −78° C. After stirring for 2 hours at this temperature and for 16 hours at ambient temperature, the reaction medium is diluted in Et₂O and then washed with a solution of NH₄Cl. The organic phase is then dried over Na₂SO₄, filtered, concentrated and purified on a silica column (2/8 v/v EtOAc/PE) to give a beige oil (300 mg, 60%).

Rf: 0.55 (3/7 v/v EtOAc/PE).

MS (ESI⁺/MeOH) m/z: 484.45 [M+Na]⁺.

¹H NMR (400.13 MHz, CD₃OD) δ (ppm): 3.27 (s, 3H, —OCH₃); 3.71 (t, 1H, J₄₋₃=J₄₋₅=9.4 Hz, H₄); 3.75 (dd, 1H, J₂₋₁=1.8 Hz, J₂₋₃=3.0 Hz, H₂); 3.84 (dd, 1H, J₃₋₂=3.2 Hz, J₃₋₄=9.2 Hz, H₃); 3.98 (m, 1H, H₅); 4.57-4.81 (m, 6H, Hₐ); 4.68 (d, 1H, J₁₋₂=1.6 Hz, H₁); 5.24, 5.27 (2 m, 1H, H₇ₐ); 5.40, 5.45 (2 m, 1H, H_{7b}); 5.99 (m, 1H, H₆); 4.73 (d, 2H, J=12.4 Hz, Hₐ); 7.21-7.35 (m, 15H, H_{c,d,e}).

¹³C NMR (100.62 MHz, CD₃OD) δ (ppm): 54.70 (—OCH₃); 72.40, 72.80 and 75.11 (3Cₐ); 72.83 (C₅); 74.80 (C₂); 78.73 (C₄); 79.82 (C₃); 99.12 (C₁); 118.12 (C₇); 127.49-128.31 (C_{c,d,e}); 135.49 (C₆); 138.31, 138.48, 138.59 (C_{b}).

### 6) SYNTHESIS OF 6-(METHYL 2,3,4-TRI-O-BENZYL-6-DEOXY-α-D-MANNO-PYRANOSIDE) BORONIC ACID (Compound 6e)

100 mg of methyl 2,3,4-tri-O-benzyl-6-deoxy-6-allyl-α-D-mannopyranoside (0.22 mmol, 1 eq.) are dissolved in 2 ml of pentane, before adding, under argon at a temperature of −78° C., 36 μl of boron tribromide (0.22 mmol, 1 eq.) and 34 μl of triethylsilane (0.22 mmol, 1 eq.). Stirring is maintained for 3 hours at this temperature, and then for 15 minutes at ambient temperature. 17 mg of sodium hydroxide (0.44 mmol, 2 eq.) are then added to the reaction mixture, which is then stirred for 30 minutes. The solution is diluted in EtOAc, then washed with water. The organic phase is dried over Na₂SO₄, filtered, concentrated and then purified on a silica column (2/8 v/v EtOAc/PE) to give a white oil (100 mg, 91%).

Rf: 0.34 (5/5 v/v EtOAc/PE).

MS (ESI⁺/MeOH) m/z: 529.87 [M+Na]⁺.

¹H NMR (400.13 MHz, CD₃OD) δ (ppm): 3.33 (dd, 1H, J₆ₐ₋₅=7.6 Hz, J_{6a-6b}=10.0 Hz, H₆ₐ); 3.38 (s, 3H, H_{—OCH3}); 3.52 (m, 1H, H₅); 5.57 (dd, 1H, J_{6b-5}=2.4 Hz, J_{6b-6a}=10.0 Hz, H_{6b}); 3.78 (t, 1H, J₄₋₃=J₄₋₅=9.2 Hz, H₄); 3.80 (m, 1H, H₂); 4.61-5.01 (m, 6H, Hₐ); 4.76 (d, 1H, J₁₋₂=1.6 Hz, H₁); 7.26-7.41 (m, 15H, H_{c,d,e}).

¹³C NMR (100.62 MHz, CD₃OD) δ (ppm): 7.04 (C₆); 55.02 (C_{—OCH3}); 71.41 (C₅); 72.07, 72.70 and 75.37 (3Cₐ); 74.58 (C₂); 78.58 (C₄); 79.90 (C₃); 99.03 (C₁); 127.60-128.43 (C_{c,d,e}); 138.17, 138.22 and 138.26 (3C_{b}).

### 7) SYNTHESIS OF 6-(METHYL 6-DEOXY-α-D-MANNOPYRANOSIDE) BORONIC ACID (Compound 7e)

100 mg of boronate (0.42 mmol, 1 eq.) are placed in solution with Amberlyst 15-H resins in 4 ml of a mixture of MeOH/THF (1/1 v/v) according to the same protocol as for {1-[(6,7-di-deoxy-α-D-mannoheptopyranosyl)uronic acid]ethyl-1H-1,2,3-triazol-4-yl}methyl-[O-(1-pent-5-yl)-O-hexa(ethylene glycol)](compound 13a) (Example 1). A whitish oil is obtained (0.40 mg, 85%).

Rf: 0.52 (5/5 v/v IPrOH/NH₄Cl).

MS (ESI⁺/MeOH) m/z: 259.37 [M+Na]⁺.

¹H NMR (400.13 MHz, D₂O) δ (ppm): 1.57 (m, 1H, H₇ₐ); 1.88 (m, 1H, H_{7b}); 2.68 (m, 1H, H₆ₐ); 2.78 (m, 1H, H_{6b}); 3.25 (s, 3H, H_{—OCH3}); 3.36 (t, 1H, J₄₋₅=J₄₋₃=9.6 Hz, H₄); 3.45 (td, 1H, J₅₋₆ₐ=J₅₋₄=9.4 Hz, J_{5-6b}=2.7 Hz, H₅); 3.57 (dd, 1H, J₃₋₄=9.4 Hz, J₃₋₂=3.5 Hz, H₃); 3.79 (dd, 1H, J₂₋₃=3.4 Hz, J₂₋₁=1.7 Hz, H₂); 4.57 (s, 1H, H₁).

¹³C NMR (100.62 MHz, D₂O) δ (ppm): 32.90 (C₇); 37.72 (C₆); 55.16 (C_{—OCH3}); 70.26 (C₂); 70.58 (C₄); 70.88 (C₃); 71.04 (C₅); 101.23 (C₁).

The scheme for synthesis of the compound of formula (III) (compound 7e) is given in the appended FIG. 6.

### EXAMPLE 4

### Preparation of a Compound of Formula (III)

### 1) SYNTHESIS OF METHYL 2,3,4-TRI-O-ACETYL-6-MONOMETHOXYTRITYL-α-D-MANNOPYRANOSIDE (Compound 1f)

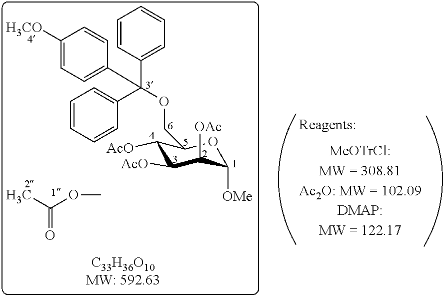

6 g (30.86 mmol-1 eq.) of methyl α-D-mannopyranoside and 1.13 g (9.26 mmol-0.3 eq.) of DMAP are dissolved in 60 ml of pyridine. 14.2 g (46.30 mmol-1.5 eq.) of monomethoxytrityl chloride are added to the mixture in fractions. The tritylation reaction lasts 1.5 hours, and is monitored by TLC (95/5 v/v CH₂Cl₂/MeOH). 13.15 ml (137.97 mmol-4.5 eq.) of acetic anhydride are then added to the reaction medium. The acetylation is monitored by TLC (7/3 v/v Et₂O/PE). After 3 hours of reaction, the pyridinium salts are filtered off and the reaction mixture is diluted in 250 ml of EtOAc. The organic phase is washed successively with a 2N solution of HCl (to pH=1), a 5% solution of NaHCO₃, and distilled water, and then dried over Na₂SO₄, filtered and concentrated. The product is then purified by silica gel chromatography with an elution gradient (3/7 v/v Et₂O/PE to 5/5 v/v Et₂O/PE) to give a white foam.

Physical appearance: White foam.

Yield: 90%.

Rf: 0.62 (7/3 v/v Et₂O/PE).

MS: (ESI⁺/MeOH) m/z: 615.2 [M+Na]⁺.

¹H NMR (400.13 MHz, CDCl₃) δ (ppm): 1.61, 1.81 and 2.02 (3 s, 9H, H_{2′}); 3.07 (m, 2H, H₆ₐ and H_{6b}); 3.32 (s, 3H, —OCH₃); 3.62 (s, 3H, H_{4′}); 3.76 (m, 1H, H₅); 4.62 (d, 1H, ³J_{H1-H2}=1.7 Hz, H₁); 5.09-5.17 (m, 3H, H₂, H₃ and H₄); 6.67-7.22 (m, 14H, 14H_{Ph}).

¹³C NMR (100.62 MHz, CDCl₃) δ (ppm): 21.0, 21.1 and 21.3 (3C, C_{2′}); 55.4 (1C, —OCH₃); 55.6 (1C, C_{4′}); 62.9 (1C, C₆); 67.1 (1C, C₄); 69.8 (1C, C₃); 70.2 (1C, C₂); 70.5 (1C, C₅); 86.8 (1C, C_{3′}); 98.7 (1C, C₁); 113.5, 127.3, 128.2, 128.3, 128.9 and 130.8 (14C, CH_{Ph}); 135.9, 144.7 and 144.8 (3C, C_{IVPh}); 155.0 (1C, C_{IVPh-}OCH₃); 169.8, 170.4 and 170.6 (3C, C_{1′}).

### 2) SYNTHESIS OF METHYL 2,3,4-TRI-O-ACETYL-α-D-MANNOPYRANOSIDE (Compound 2f)

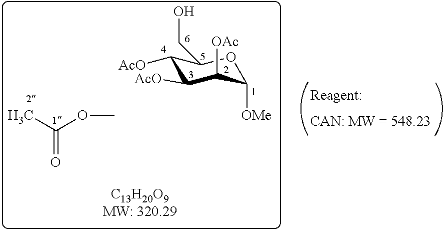

9.16 g (15.45 mmol-1 eq.) of methyl 2,3,4-tri-O-acetyl-6-monomethoxytrityl-α-D-mannopyranoside are dissolved in 150 ml of a mixture of CH₃CN/H₂O (95/5 v/v). 847 mg (1.55 mmol-0.1 eq.) of CAN are added, and then the mixture is heated at a temperature of 60° C.

The reaction is monitored by TLC (7/3 v/v Et₂O/PE) and lasts 1 hour. The reaction medium is then diluted with CH₂Cl₂. The organic phase is then washed twice with distilled water, and then dried over Na₂SO₄, filtered and concentrated. The product is then purified by flash chromatography on silica gel with an elution gradient (CH₂Cl₂ to 96/4 v/v CH₂Cl₂/MeOH) to give a white powder.

Physical appearance: White powder.

Yield: 90%.

Rf: 0.67 (95/5 v/v CH₂Cl₂/MeOH).

MS: (ESI⁺/MeOH) m/z: 321.4 [M+H]⁺; 343.1 [M+Na]⁺; 663.5 [2M+Na]⁺,

(ESI⁻/MeOH) m/z: 639.2 [2M−H]⁻.

¹H NMR (400.13 MHz, CDCl₃) δ (ppm): 1.75, 1.83 and 1.90 (3 s, 9H, H_{2′}); 2.48 (s, 1H, OH); 3.18 (s, 3H, —OCH₃); 3.41 (dd, 1H, ³J_{H6a-H5}=4.5 Hz, ²J_{H6a-H6b}=−12.6 Hz, H₆ₐ); 3.48 (dd, 1H, ³J_{H6b-H5}=2.0 Hz, ²J_{H6b-H6a}=−12.5 Hz, H_{6b}); 3.54 (ddd, 1H, ³J_{H5-H6a}=4.5 Hz, ³J_{H5-H6b}=2.3 Hz, ³J_{H5-H4}=9.9 Hz, H₅); 4.50 (d, 1H, ³J_{H1-H2}=1.6 Hz, H₁); 5.00 (t, 1H, ³J_{H4-H3}=³J_{H4-H5}=10.0 Hz, H₄); 5.01 (dd, 1H, ³J_{H2-H1}=1.8 Hz, ³J_{H2-H3}=3.5 Hz, H₃); 5.13 (dd, 1H, ³J_{H3-H2}=3.4 Hz, ³J_{H3-H4}=10.2 Hz, H₃).

¹³C NMR (100.62 MHz, CDCl₃) δ (ppm): 20.9, 21.0 and 21.1 (3C, C_{2′}); 55.5 (1C, —OCH₃); 61.6 (1C, C₆); 66.7 (1C, C₄); 69.3 (1C, C₃); 69.2 (1C, C₂); 70.9 (1C, C₅); 98.1 (1C, C₁); 170.2, 170.4 and 171.0 (3C, C_{1′}).

### 3) SYNTHESIS OF METHYL 6-O-HYDROGENOPHOSPHONATE-α-D-MANNO-PYRANOSIDE (Compound 4f)

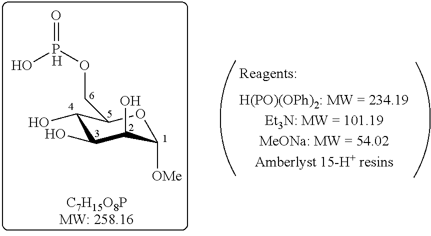

0.5 g (1.56 mmol-1 eq.) of methyl 2,3,4-tri-O-acetyl-α-D-mannopyranoside (compound 3f) are dissolved in 10 ml of distilled pyridine. 2.1 ml (10.92 mmol-7 eq.) of diphenyl phosphite are added dropwise to the mixture at ambient temperature. After stirring for minutes, 4 ml of a mixture of Et₃ N/H₂O (1/1 v/v) are added to the reaction medium, and then the mixture is kept stirring for a further 30 minutes. The reaction is monitored by TLC (95/5 v/v iPrOH/NH₄OH). The reaction mixture is then concentrated, and then diluted in 50 ml of CH₂Cl₂. The organic phase is washed three times with a saturated solution of NaHCO₃, washed with distilled water, and then dried over Na₂SO₄, filtered and concentrated. During this step, all of the starting sugar is used up. 28 mg (0.46 mmol-0.3 eq.) of MeONa are added to the reaction medium, and then diluted again in 10 ml of anhydrous MeOH. The reaction is monitored by TLC (91/v/v iPrOH/NH₄OH) and lasts 30 minutes. The reaction medium is then neutralized with Amberlyst 15-H⁺ acid resins. The resins are then filtered off, then rinsed with MeOH. The crude obtained after concentration is purified by silica gel column chromatography with an elution gradient (iPrOH to 91/v/v iPrOH/NH₄OH).

Physical appearance: Light beige powder.

Yield: 93%.

Rf: 0.24 (9/1 v/v iPrOH/NH₄OH).

MS (ESI⁺/MeOH) m/z: 259.2 [M+H]⁺; 281.1 [M+Na]⁺; 539.2 [2M+Na]⁺,

(ESI⁻/MeOH) m/z: 257.4 [M−H]⁻.

¹H NMR (400.13 MHz, CD₃OD) δ (ppm): 3.23-3.26 (m, 1H, H₆ₐ); 3.32 (s, 3H, —OCH₃); 3.32-3.36 (m, 1H, H5); 3.45 (t, 1H, ³J_{H4-H3}=³J_{H4-H5}=9.4 Hz, H4); 3.5 (dd, 1H, ³J_{H6b-H5}=2.2 Hz, ²J_{H6b-H6a}=−10.9 Hz, H_{6b}); 3.65 (dd, 1H, ³J_{H3-H2}=3.4 Hz, J_{H3-H4}=9.5 Hz, H₃); 3.82 (dd, 1H, ³J_{H1-H2}=1.7 Hz, ³J_{H2-H3}=3.4 Hz, H2); 6.10 (d, 1H, ³J_{H1-H2}=1.5 Hz, H1).

¹³C NMR (100.62 MHz, CD₃OD) δ (ppm): 6.3 (1C, C₆); 55.0 (1C, —OCH₃); 69.8 (1C, C₂); 70.1 (1C, C₃); 70.6 (1C, C₄); 71.5 (1C, C₅); 101.1 (1C, C₁).

³¹P NMR (162 MHz, CD₃OD) δ (ppm): 9.23 (s, H(PO)OH).

The scheme for synthesis of the compound of formula (III) (compound 4f) is given in the appended FIG. 7.

### EXAMPLE 5

### Preparation of a Pyrophosphonate Derivative of Formula (III)

### 1) SYNTHESIS OF METHYL 2,3-O—ISOPROPYLIDENE-4,6-O-(CYCLOSULFATE)-α-D-MANNOPYRANOSIDE (Compound 1g)

3.79 g of methyl 2,3-O-isopropylidene-α-D-mannopyranoside (16.18 mmol, 1 eq.), 6.75 ml of triethylamine (48.54 mmol, 3 eq.) and 1.3 ml of thionyl chloride (17.80 mmol, 1.1 eq.) are reacted in 75 ml of CH₂Cl₂ according to the same protocol as for 2′-azidoethyl-2,3-O-isopropylidene-4,6-O-(cyclosulfate)-α-D-mannopyranose (compound 5a) (Example 1). The crude sulfite (16.18 mmol, 1 eq.), 3.8 g of sodium metaperiodate (17.80 mmol, 1.1 eq.), 20 ml of water and 14 mg of ruthenium chloride (0.06 mmol, 0.004 eq.) are then reacted in 60 ml of a solution of CH₂Cl₂/CH₃CN (1/1 v/v) according to the same protocol.

Rf: 0.48 (3/7 v/v EtOAc/PE).

MS (ESI⁺/MeOH) m/z: 297.65 [M+H]⁺; 319.23 [M+Na]⁺.

### 2) SYNTHESIS OF METHYL 6,7-DIDEOXY-DIMETHOXYPHOSPHINYL-2,3-O-ISOPROPYLIDENE-4-(SODIUM SULFATE)-α-D-MANNOPYRANOSIDE (Compound 2g)

In a two-necked flask, 3.5 g of dimethylmethylphosphonate (28.37 mmol, 2 eq.), 3 drops of 1,1-diphenylethylene (colored indicator) and 10 ml of DMPU (40.53 mmol, 4 eq.) are dissolved in 20 ml of anhydrous THF, under an argon atmosphere. The two-necked flask is immersed in a bath at a temperature of −80° C. for 5 minutes. An excess of BuLi is then added dropwise until a persistent red coloration is obtained. 6 g of cyclosulfate (20.27 mmol, 1 eq.) previously dissolved in 40 ml of anhydrous THF are added dropwise. The solution turns yellow after the addition of a few drops. The bath is then kept at a temperature of −70/−80° C. for 3 hours, and then at ambient temperature for 14 hours. The mixture is then diluted in CH₂Cl₂, and then the product is extracted by washing with water. The aqueous phase is washed with CH₂Cl₂, before being lyophilized. The brown oil obtained is purified by silica gel chromatography (9/1 v/v CH₂Cl₂/MeOH) to give a yellow oil (1.84 g, 20%).

Rf: 0.28 (9/1 v/v CH₂Cl₂/MeOH).

MS (ESI⁺/MeOH) m/z: 465.15 [M+Na]⁺

(ESI⁻/MeOH) m/z: 419.18 [M−Na]⁻.

¹H NMR (400.13 MHz, CD₃OD) δ (ppm): 1.33, 1.51 (2 s, 6H, H₁₀); 1.89 and 2.17 (2 m, 4H, H₆ and H₇); 3.35 (s, 3H, —OCH₃); 3.62 (m, 1H, H₅); 3.74 and 3.77 (2 s, 6H, H₈); 4.10 (d, 1H, J₂₋₁=J₂₋₃=5.2 Hz, H₂); 4.23 (m, 2H, H₃ and H₄); 4.83 (s, 1H, H₁).

¹³C NMR (100.62 MHz, CD₃OD) δ (ppm): 21.00 and 25.28 (C₆ and C₇); 26.30 and 27.90 (C₁₀); 53.09 and 53.16 (C₈); 55.43 (—OCH₃); 68.60 (C₅); 76.79 (C₂); 77.93 and 78.56 (C₃ and C₄); 99.54 (C₁); 110.41 (C₉).

³¹P NMR (81.02 MHz, CD₃OD) δ (ppm): 36.31.

### 3) SYNTHESIS OF METHYL 6,7-DIDEOXY-PHOSPHINYL-α-D-MANNO-PYRANOSIDE (Compound 3g)

1.8 g of methyl 6,7-dideoxy-dimethoxyphosphinyl-2,3-O-isopropylidene-4-(sodium sulfate)-α-D-mannopyranoside (4.07 mmol, 1 eq.) are first of all reacted in 15 ml of a mixture of CH₃CN/H₂O (8/2 v/v) according to the protocol described for 2′-azidoethyl-2,3-O-isopropylidene-α-D-mannopyranose (compound 4a) (Example 1). 1.1 g of the product obtained, a yellow oil (3.67 mmol, 1 eq.), are reacted with 2.9 ml of pyridine (36.77 mmol, 10 eq.) and 2.42 ml of trimethylsilane bromide (18.33 mmol, 5 eq.) in 12 ml of dichloromethane, under an argon atmosphere. After stirring for 8 hours at ambient temperature, the mixture is concentrated and 10 ml of 0.1N sodium hydroxide are then added. The stirring is maintained for 30 minutes. The traces of pyridine are removed via three extractions with Et₂O, and then the aqueous phase is acidified with a 1N HCl solution, before extraction three times with Et₂O. The organic phases are combined, dried over Na₂SO₄, filtered and concentrated, to give a colorless oil (708 mg, 71%).

Rf: 0.18 (8/2 v/v CH₂Cl₂/MeOH).

MS (ESI⁺/MeOH) m/z: 295.12 [M+Na]⁺.

¹H NMR (400.13 MHz, CD₃OD) δ (ppm): 1.94 and 2.17 (2 m, 4H, H₆ and H₇); 3.36 (s, 3H, —OCH₃); 7.20 (m, 1H, H₅); 3.87 (m, 2H, H₂ and H₃); 4.32 (t, 1H, J₄₋₃=J₄₋₅=9.2 Hz, H₄); 4.64 (s, 1H, H₁).

¹³C NMR (100.62 MHz, CD₃OD) δ (ppm): 20.93 and 25.03 (C₆ and C₇); 55.52 (—OCH₃); 70.33 (H₅); 71.58 and 71.85 (C₂ and C₃); 78.48 (C₄); 102.23 (C₁).

³¹P NMR (81.02, CD₃OD) δ (ppm): 36.66.

### 4) SYNTHESIS OF METHYL 6,7-DIDEOXY-PHOSPHINYL-2,3,4-O-ACETYL-α-D-MANNOPYRANOSIDE (Compound 4g)

1.1 g of methyl 6,7-dideoxy-phosphinyl-α-D-mannopyranoside (2.74 mmol, 1 eq.) and 2 ml of acetic anhydride (10.9 mmol, 4 eq.) are reacted in 20 ml of pyridine. After stirring for 1 hour at ambient temperature, the solution is diluted in CH₂Cl₂, then washed with a 1N HCl solution and water. The organic phase is dried over Na₂SO₄, filtered and concentrated, to give a brown oil (850 mg, 64%).

Rf: 0.15 (8/2 v/v CH₂Cl₂/MeOH).

MS (ESI⁺/MeOH) m/z: 421.87 [M+Na]⁺.

¹H NMR (400.13 MHz, CD₃OD) δ (ppm): 1.90 and 2.21 (2 m, 4H, H₆ and H₇); 2.01 and 2.11 (2 s, 6H, H_{b}); 3.41 (s, 3H, —OCH₃); 3.62 (m, 1H, H₅); 4.41 (t, 1H, J₄₋₃=J₄₋₅=9.8 Hz, H₄); 4.67 (s, 1H, H₁); 5.12 (m, 1H, H₂); 5.26 (dd, 1H, J₃₋₂=3.4 Hz, J₃₋₄=9.8 Hz, H₃).

¹³C NMR (100.62 MHz, CD₃OD) δ (ppm): 19.62 and 25.18 (C₆ and C₇); 20.73 and 20.85 (C_{b}); 55.66 (—OCH₃); 70.63, 71.56, 72.87 (H₂, C₃ and C₅); 75.73 (C₄); 99.68 (C₁); 171.75, 172.26 (Cₐ).

³¹P NMR (81.02, CD₃OD) δ (ppm): 36.31.

### 5) SYNTHESIS OF METHYL 6,7-DIDEOXY-PYROPHOSPHINYL-2,3,4-O-ACETYL-α-D-MANNOPYRANOSIDE (Compound 5g)

700 mg of methyl 6,7-dideoxy-phosphinyl-2,3,4-O-acetyl-α-D-mannopyranoside (0.15 mmol, 1 eq.) are dissolved in 8 ml of methanol, before adding dibutylamine (0.15 mmol, 1 eq.). The mixture is left to stir at ambient temperature for 30 minutes. The solvent is then evaporated off, and then co-evaporated off with anhydrous pyridine in order to remove any trace of water. The phosphonic monosalt of dibutylamino obtained is dissolved in 7 ml of anhydrous THF, and then diphenyl chlorophosphate (0.15 mmol, 1 eq.) and dibutylamine (4.39 mmol, 3 eq.) are successively added. The mixture is kept stirring at ambient temperature under an argon atmosphere for 2 hours.

In the same way, the monosalt of dibutylamine orthophosphate is prepared: orthophosphoric acid (4.39 mmol, 3 eq.) is dissolved in 8 ml of methanol, and then dibutylamine (4.39 mmol, 3 eq.) is subsequently added. After stirring for 20 minutes at ambient temperature, the traces of pyridine are removed by co-evaporation with anhydrous pyridine. The monosalt of dibutylammonium orthophosphate (4.39 mmol, 3 eq.) is dissolved in 8 ml of anhydrous pyridine, and then activated phosphonic anhydride is slowly added. The solution is kept stirring at ambient temperature under an argon atmosphere for 15 hours. The solvents are then evaporated off, and the oil obtained is purified by silica gel chromatography (9/1 v/v iPrOH/NH₄Cl) to give a transparent oil (470 mg, 50%).

Rf: 0.12 (8/2 v/v CH₂Cl₂/MeOH).

MS (ESI⁺/MeOH) m/z: 421.87 [M+Na]⁺.

¹H NMR (400.13 MHz, CD₃OD) δ (ppm): 1.74 and 2.00 (2 m, 4H, H₆ and H₇); 2.02 and 2.10 (2 s, 6H, H_{b}); 3.40 (s, 3H, —OCH₃); 3.58 (m, 1H, H₅); 4.43 (t, 1H, J₄₋₃=J₄₋₅=9.8 Hz, H₄); 4.69 (s, 1H, H₁); 5.13 (m, 1H, H₂); 5.28 (dd, 1H, J₃₋₂=3.4 Hz, J₃₋₄=9.8 Hz, H₃).

¹³C NMR (100.62 MHz, CD₃OD) δ (ppm): 19.56 and 25.01 (C₆ and C₇); 20.73 and 20.85 (C_{b}); 55.69 (—OCH₃); 70.63, 71.58, 72.87 (H₂, C₃ and C₅); 75.71 (C₄); 99.65 (C₁); 171.75, 172.26 (Cₐ).

³¹P NMR (81.02, CD₃OD) δ (ppm): −9.9 and 8.4.

### 6) SYNTHESIS OF METHYL 6,7-DIDEOXY-PYROPHOSPHINYL-α-D-MANNO-PYRANOSIDE (Compound 6g)

470 mg of methyl 6,7-dideoxy-phosphinyl-α-D-mannopyranoside (0.89 mmol, 1 eq.) are deprotected according to the protocol described for 2′-azidoethyl-α-D-mannopyranose (compound 3a) (Example 1) in 8 ml of methanol and in the presence of 190 mg of sodium methanolate (3.55 mmol, 4 eq.), to give a white oil (297 mg, 80%).

Rf: 0.20 (5/5 v/v IPrOH/NH₄Cl).

MS (ESI⁺/MeOH) m/z: 441.57 [M+Na]⁺.

¹H NMR (400.13 MHz, D₂O) δ (ppm): 1.97 (m, 1H, H₇ₐ); 2.36 (m, 1H, H_{7b}); 2.99 (m, 1H, H₆ₐ); 3.13 (m, 1H, H_{6b}); 3.37 (s, 3H, —OCH₃); 3.78 (m, 1H, H₅); 3.89-3.96 (m, 2H, H₂ and H₃); 4.45 (t, 1H, J₄₋₅=J₄₋₃=9.4 Hz, H₄); 4.70 (s, 1H, H₁).

¹³C NMR (100.62 MHz, D₂O) δ (ppm): 26.81 (C₇); 47.60 (C₆); 55.43 (—OCH₃); 69.16 (C₅); 69.94 (C₃); 70.44 (C₂); 79.03 (C₄); 100.99 (C₁).

³¹P NMR (81.02, CD₃OD) δ (ppm): −9.8 and 8.5.

The scheme for synthesis of the pyrophosphonate derivative of formula (III) (compound 6g) is given in the appended FIG. 8.

### EXAMPLE 6

### Preparation of a Pyrophosphate Derivative of Formula (III)

Compounds 1h, 2h and 3h are prepared according to the procedure described above (cf. synthesis of compounds 1e, 2e and 3e of example 3).

### 1) SYNTHESIS OF METHYL 6-DEOXY-6-(ETHYL PHOSPHATE)-2,3,4-TRI-O-BENZYL-α-D-MANNOPYRANOSIDE (Compound 4h)

100 mg of sugar (0.22 mmol, 1 eq.) and 750 mg of diethyldiethylphosphoramidite (0.22 mmol, 1 eq.) are dissolved in 2 ml of THF, before adding, under an argon atmosphere, 290 μl of 1H-tetrazole dropwise. After stirring for 4 hours at ambient temperature, 600 mg of meta-chloroperbenzoic acid (mCPBA) (3.47 mmol, 1.6 eq.), dissolved beforehand in 6 ml of CH₂Cl₂, are added to the mixture at a temperature of −78° C. Stirring is maintained at this temperature for 10 minutes, and then at ambient temperature for 10 minutes. The reaction mixture is diluted with EtOAc, then washed with a saturated solution of NaHCO₃. The organic phase is dried over Na₂SO₄, filtered, concentrated, and then purified on a silica column (2/8 v/v EtOAc/PE) to give a transparent oil.

Rf: 0.45 (9/1 v/v CH₂Cl₂/MeOH).

MS (ESI⁺/MeOH) m/z: 623.13 [M+Na]⁺.

¹H NMR (400.13 MHz, CDCl₃) δ (ppm): 1.21 (t, 6H, J₈₋₇=7.1 Hz, H₈); 2.80 (m, 1H, H₆ₐ); 2.93 (m, 1H, H_{6b}); 3.29 (s, 3H, —OCH₃); 3.78 (td, 1H, J₅₋₄=J₅₋₆ₐ=9.3 Hz, J_{5-6b}=2.6 Hz, H₅); 4.03 (m, 4H, H₇); 4.53 (s, 1H, H₁); 4.55-4.64 (m, 6H, Hₐ); 5.00 (t, 1H, J₄₋₅=J₄₋₃=9.9 Hz, H₄); 5.07 (dd, 1H, J₂₋₃=3.3 Hz, J₂₋₁=1.7 Hz, H₂); 5.15 (dd, 1H, J₃₋₂=3.4 Hz, J₃₋₄=10.0 Hz, H₃); 7.17-7.31 (m, 15H, H_{c,d,e}).

¹³C NMR (100.62 MHz, CDCl₃) δ (ppm): 16.42 and 16.58 (C₈); 32.63 (C₆); 55.76 (—OCH₃); 64.03 and 64.10 (H₇); 69.31 (C₃ and C₄); 69.97 (C₂); 70.58 (C₅); 98.84 (C₁); 72.33, 73.01 and 75.34 (Cₐ); 127.64-128.56 (C_{c,d,e}); 138.30, 138.41 and 138.55 (C_{b}).

³¹P NMR (81.02 CDCl₃) δ (ppm): 28.0.

### 2) SYNTHESIS OF METHYL 6-DEOXY-6-PHOSPHATE-2,3,4-TRI-O-BENZYL-α-D-MANNOPYRANOSIDE (Compound 5h)

Compound 5h is prepared according to the procedure described above (cf. example 5, synthesis of methyl 6,7-dideoxy-phosphinyl-2,3,4-O-acetyl-α-D-mannopyranoside).

Rf: 0.23 (9/1 v/v CH₂Cl₂/MeOH).

MS (ESI⁺/MeOH) m/z: 567.34 [M+Na]⁺.

¹H NMR (400.13 MHz, CDCl₃) δ (ppm): 2.96 (m, 1H, H₆ₐ); 3.23 (m, 1H, H_{6b}); 3.28 (s, 3H, —OCH₃); 3.65 (td, 1H, J₅₋₄=J₅₋₆ₐ=9.3 Hz, J_{5-6b}=2.6 Hz, H₅); 4.51 (s, 1H, H₁); 4.54-4.64 (m, 6H, Hₐ); 5.04 (t, 1H, J₄₋₅=J₄₋₃=9.9 Hz, H₄); 5.04 (dd, 1H, J₂₋₃=3.3 Hz, J₂₋₁=1.7 Hz, H₂); 5.17 (dd, 1H, J₃₋₂=3.4 Hz, J₃₋₄=10.0 Hz, H₃); 7.18-7.32 (m, 15H, H_{c,d,e}).

¹³C NMR (100.62 MHz, CDCl₃) δ (ppm): 32.61 (C₆); 55.78 (—OCH₃); 69.33 (C₃ and C₄); 69.89 (C₂); 70.56 (C₅); 98.84 (C₁); 72.36, 73.05 and 75.34 (Cₐ); 127.64-128.53 (C_{c,d,e}); 138.33, 138.48 and 138.65 (C_{b}).

³¹P NMR (81.02 CDCl₃) δ (ppm): 22.5.

### 3) SYNTHESIS OF METHYL 6-DEOXY-6-PYROPHOSPHATE-2,3,4-TRI-O-BENZYL-α-D-MANNOPYRANOSIDE (Compound 6h)

100 mg of methyl 6-deoxy-6-phosphate-2,3,4-tri-O-benzyl-α-D-mannopyranoside (0.52 mmol, 1 eq.) are dissolved in 3 ml of anhydrous THF, and then 85 μl of pyridine (1.03 mmol, 2 eq.) and 52 μl of POCl₃ (0.57 mmol, 1.1 eq.) are added at a temperature of 0° C. under an argon atmosphere. Stirring is maintained at a temperature of 0° C. for 4 hours, a few ml of a saturated solution of NaHCO₃ are added, still at a temperature of 0° C., and the mixture is stirred for a further 15 minutes. The reaction medium is subsequently lyophilized, and then purified on a silica column (9/1 v/v CH₂Cl₂/MeOH) to give a whitish oil (35 mg, 30%).

Rf: 0.12 (8/2 v/v CH₂Cl₂/MeOH).

MS (ESI⁺/MeOH) m/z: 647.78 [M+Na]⁺.

¹H NMR (400.13 MHz, CDCl₃) δ (ppm): 2.96 (m, 1H, H₆ₐ); 3.23 (m, 1H, H_{6b}); 3.28 (s, 3H, —OCH₃); 3.65 (td, 1H, J₅₋₄=J₅₋₆ₐ=9.3 Hz, J_{5-6b}=2.6 Hz, H₅); 4.51 (s, 1H, H₁); 4.54-4.64 (m, 6H, Hₐ); 5.04 (t, 1H, J₄₋₅=J₄₋₃=9.9 Hz, H₄); 5.04 (dd, 1H, J₂₋₃=3.3 Hz, J₂₋₁=1.7 Hz, H₂); 5.17 (dd, 1H, J₃₋₂=3.4 Hz, J₃₋₄=10.0 Hz, H₃); 7.18-7.32 (m, 15H, H_{c,d,e}).

¹³C NMR (100.62 MHz, CDCl₃) δ (ppm): 32.61 (C₆); 55.78 (—OCH₃); 69.33 (C₃ and C₄); 69.89 (C₂); 70.56 (C₅); 98.84 (C₁); 72.36, 73.05 and 75.34 (Cₐ); 127.64-128.53 (C_{c,d,e}); 138.33, 138.48 and 138.65 (C_{b}).

³¹P NMR (81.02 CDCl₃) δ (ppm): −6.78 and 7.34.

### 4) SYNTHESIS OF METHYL 6-DEOXY-6-PYROPHOSPHATE-α-D-MANNO-PYRANOSIDE (Compound 7h)

Compound 7h is prepared according to the procedure described above (cf. example 5, synthesis of methyl 6,7-dideoxy-pyrophosphinyl-2,3,4-O-acetyl-α-D-mannopyranoside).

Rf: 0.12 (7/3 v/v iPrOH/NH₄Cl).

MS (ESI⁺/MeOH) m/z: 377.91 [M+Na]⁺.

¹H NMR (400.13 MHz, CD₃OD) δ (ppm): 3.44 (s, 3H, H_{—OCH3}); 3.64 (m, 1H, H₅); 3.67 (t, 1H, J₄₋₃=J₄₋₅=10.0 Hz, H₄); 3.77 (m, 1H, H₆ₐ); 3.80 (d, 1H, J₂₋₁=J₂₋₃=5.6 Hz, H₂); 3.91 (d, 1H, J₁₋₂=1.6 Hz, H₁); 3.96 (dd, 1H, J_{6b-5}=1.8 Hz, J_{6b-6a}=9.8 Hz, H_{6b}); 3.97 (dd, 1H, J₃₋₂=1.6 Hz, J₃₋₄=3.2 Hz, H₃).

¹³C NMR (100.62 MHz, CD₃OD) δ (ppm): 58.59 (C₆); 64.42 (C₄); 67.57 (C₃); 68.19 (C₂); 70.19 (C₅); 98.51 (C₁).

³¹P NMR (81.02 CDCl₃) δ (ppm): −6.53 and 8.62.

The scheme for synthesis of the pyrophosphate derivative of formula (III) is given in the appended FIG. 9.

### EXAMPLE 7

### Preparation of a Compound of Formula (II)

### 1) SYNTHESIS OF METHYL 6-DEOXY-6-AZIDO-D-MANNOPYRANOSYL-(1,6)-6-DEOXY-6-AZIDO-D-MANNOPYRANOSYL-(1,4)-D-MANNOPYRANOSIDE

12 g of trichloroacetimidate (0.03 mol, 3 eq.) are added to 2.35 g of methyl mono-2,3-isopropylidene-mannopyranoside (0.01 mol, 1 eq.) dissolved in 40 ml of anhydrous THF. The solution is then cooled to a temperature of 0° C., before 30 ml (0.28 mol) of BF₃Et₂O are added dropwise under an argon atmosphere. The reaction mixture is then maintained at ambient temperature and left to stir overnight. Once the reaction is complete, the reaction mixture is washed successively with twice 80 ml of a saturated aqueous solution of NaHCO₃, then with twice 80 ml of distilled water. The organic phase is dried with Na₂SO₄, filtered and evaporated under reduced pressure. The residue obtained is chromatographed on silica gel using a mixture of CH₂Cl₂/MeOH as eluent. The product obtained is a white powder (5 g, yield 80%).

Rf: 0.5 (8/2 v/v CH₂Cl₂/MeOH).

MS (ESI⁺/MeOH) m/z: 661.7 [M+Na]⁺; 661.7 [M+H]⁺.

3.3 g of trisaccharide (5 mmol, 1 eq.) and 2.1 ml of triethylamine (15 mmol, 3 eq.) are dissolved in 25 ml of CH₂Cl₂. The round-bottomed flask is placed in an ice bath and 3.5 ml of thionyl chloride (5.5 mmol, 1.1 eq.) are slowly added. A white precipitate of triethylammonium chloride rapidly appears and the reaction mixture gradually turns yellow. After stirring for 5 minutes at a temperature of 0° C., the starting product has disappeared, and the desired sulfite is obtained. The mixture is filtered, and the organic phase is washed with distilled water, a 1N HCl solution, and then again water. It is then dried over Na₂SO₄, filtered and concentrated, to give a brown solid which is reused directly in reaction.

Formation of the Sulfate:

The crude sulfite (5 mmol, 1 eq.) is dissolved in 20 ml of a mixture of CH₂Cl₂/CH₃CN (1/1 v/v). 1.17 g of sodium metaperiodate (5.5 mmol, 1.1 eq.), 5 ml of water and three grains of ruthenium chloride are successively added. The reaction is exothermic, an NaIO₃ precipitate forms very rapidly. After stirring for 1 hour at ambient temperature, the sulfite has been used up, and the reaction mixture is filtered and diluted in 200 ml of CH₂Cl₂. The organic phase is washed with a 5% NaHCO₃ solution and distilled water, and then dried, filtered and concentrated. The solid obtained is dissolved in a minimum amount of CH₂Cl₂ and filtered off on silica. The silica is rinsed several times with CH₂Cl₂. A white solid is obtained (2.8 g, 70%).

Formation of the Protected Di-Azido:

823 mg of cyclic sulfate (0.15 mmol, 1 eq.) and 200 mg of sodium azide (0.31 mmol, 2 eq.) are dissolved in 10 ml of DMF. After stirring for 4 hours at ambient temperature, the reaction mixture is diluted in 50 ml of 5% NaHCO₃ and taken up with 100 ml of CH₂Cl₂, washed with water, dried over Na₂SO₄, and then evaporated. The powder obtained is dissolved in 10 ml of methanol, and then treated with 2 ml of Amberlite H⁺ resin. After evaporation of the solvent, 800 mg of yellow powder are isolated and chromatographed on silica gel with an elution gradient (CH₂Cl₂ to 6/4 v/v CH₂Cl₂/MeOH) to give a white powder (yield of 80%).

Rf: 0.67 (6/4 v/v CH₂Cl₂/MeOH).

MS (ESI⁺/MeOH) m/z: 688.7 [M+H]⁺; 710.7 [M+Na]⁺.

(ESI⁻/MeOH) m/z: 687.7 [M−H]⁻.

The scheme for synthesis of the compound of formula (II) exemplified is given in the appended FIG. 10.

### EXAMPLE 8

### Preparation of a Compound of Formula (III)

### 1) SYNTHESIS OF METHYL 6-DEOXY-6-IODO-α-D-MANNOPYRANOSIDE (Compound 1i)

A solution of 25 ml of anhydrous THF containing 4.90 g (19.3 mmol, 1.5 eq.) of diiodine is added, dropwise, under nitrogen, to a solution of anhydrous THF at reflux containing 100 ml of anhydrous THF containing 2.5 g (12.8 mmol-1 eq.) of methyl α-D-mannopyranoside, 5 g (19.3 mmol-1.5 eq.) of Pφ₃ and 1.75 g (25.7 mmol-2 eq.) of imidazole. After 3 hours of reaction at reflux, the mixture is cooled to ambient temperature, the imidazole salts are filtered and, after concentration of the filtrate, the reaction crude is directly purified by silica gel column chromatography (9/1 v/v CH₂Cl₂/MeOH). The product is then recrystallized from Et₂O, to give white crystals (85%).

Rf: 0.25 (9/1 v/v CH₂Cl₂/MeOH).

Pf: 118-120° C.

MS (ESI⁻/CH₃CN—H₂O—CF₃CO₃H) m/z: 305.0 [M+H]⁺, 327.0 [M+Na]⁺, 609.0 [2M+H]⁺,

(ESI⁻/CH₃CN—H₂O—CF₃CO₃H) m/z: 339.2 [M+Cl]⁺.

¹H NMR (400.13 MHz, CDCl₃) δ (ppm): 3.26 (dd, 1H, J₆ₐ₋₅=7.0 Hz, J_{6a-6b}=10.9 Hz, H₆ₐ); 3.32 (s, 3H, —OCH₃); 3.32-3.36 (m, 1H, H₅); 3.45 (t, 1H, J₄₋₃=J₄₋₅=9.4 Hz, H₄); 3.5 (dd, 1H, J_{6b-5}=2.2 Hz, J_{6b-6a}=10.9 Hz, H_{6b}); 3.65 (dd, 1H, J₃₋₂=3.4 Hz, J₃₋₄=9.5 Hz, H₃); 3.82 (dd, 1H, J₁₋₂=1.7 Hz, J₂₋₃=3.4 Hz, H₂); 6.10 (d, 1H, J₁₋₂=1.5 Hz, H₁).

¹³C NMR (100.62 MHz, CDCl₃) δ (ppm): 6.31 (C₆); 55.08 (—OCH₃); 69.86 (C₂); 70.14 (C₃); 70.66 (C₄); 71.59 (C₅); 101.10 (C₁).

[α]_{D}: +67.5 (c=1.00 g/100 ml, MeOH).

### 2) SYNTHESIS OF METHYL 6-DEOXY-6-IODO-2,3,4-TRI-O-BENZYL-α-D-MANNOPYRANOSIDE (Compound 2i)

4.37 g of methyl 6-deoxy-6-iodo-α-D-mannopyranoside (14.34 mmol, 1 eq.) are dissolved in 100 ml of anhydrous DMF, before adding 8.5 ml of benzyl bromide (71.72 mmol, 5 eq.). 1.7 g of NaH (71.72 mmol, 5 eq.) are then added in small fractions over a period of 1 hour. After 4 hours of reaction, 5 ml of MeOH are added and the mixture is diluted in the ether Et₂O before washing with water. The organic phase is rewashed several times with water, dried, and then concentrated. Purification by silica column chromatography (4/6 v/v EtOAc/PE) makes it possible to obtain the product in the form of a yellow oil (3.57 g, 44%).

Rf: 0.42 (8/2 v/v PE/EtOAc).

MS (ESI ESI⁺/CH₃CN—H₂O—CF₃CO₃H) m/z: 543.1 [M−OCH3]⁺, 592.2 [M+NH₄]⁺, 597.1 [M+Na]⁺.

¹H NMR (400.13 MHz, CDCl₃) δ (ppm): 3.31-3.43 (m, 1H, H₆ₐ); 3.38 (s, 3H, —OCH₃); 3.47-3.59 (m, 2H, H₅ and H_{6b}); 3.76-3.80 (m, 2H, H₂ and H₄); 3.90 (dd, 1H, J₃₋₂=2.9 Hz, J₃₋₄=9.2 Hz, H₃); 4.61 (s, 2H, Hₐ); 4.74 (d, 2H, J=12.2 Hz, Hₐ); 4.76 (d, 1H, J₁₋₂=1.5 Hz, H₁); 4.84 (d, 2H, J=11.0 Hz, Hₐ); 7.28-7.41 (m, 15H, 15H_{c,d,e}).

¹³C NMR (100.62 MHz, CDCl₃) δ (ppm): 7.01 (C₆); 55.05 (—OCH3); 71.48 (C₅); 72.09, 72.73 (2Cₐ); 74.56 (C₂); 75.44 (Cₐ); 78.52 (C₄); 79.87 (C₃); 99.05 (C₁); 127.66, 127.82, 128.06, 128.33, 128.40 (15C, C_{c,d,e}); 138.11 (C_{b}); 138.24 (2C_{b}).

[α]_{D}: +28.0 (c=1.00 g/100 ml, CHCl₃).

### 3) SYNTHESIS OF (3R,4R,5S,6R)-3,4,5-TRIS(BENZYLOXY)TETRAHYDRO-6-METHOXYPYR-2-ENE (Compound 3i)

1 g of methyl 6-deoxy-6-iodo-2,3,4-tri-O-benzyl-α-D-mannopyranoside (1.74 mmol, 1 eq.) and 2.6 ml of DBU (17.4 mmol, 10 eq.) are reacted under argon in 20 ml of DMF. After 3 and a half hours of reaction at 80° C., the solution is cooled to ambient temperature, diluted in EtOAc and washed with a saturated solution of NaHCO₃. The organic phase is then washed with water, dried, concentrated and purified by silica column chromatography (2/8 v/v EtOAc/PE) to give a brown oil (430 mg, 56%).

Rf: 0.34 (5/5 v/v EtOAc/PE).

MS (ESI⁺/MeOH): m/z 469.34 [M+Na]⁺.

¹H NMR (400.13 MHz, CDCl₃) δ (ppm): 3.62 (s, 3H, —OCH₃); 3.99 (dd, 1H, J₂₋₁=2.6 Hz, J₂₋₃=8.2 Hz, H₂); 4.02 (m, 2H, H₄ and 1Hₐ); 4.17 (d, 2H, J=10.2 Hz, Hₐ); 4.34-4.62 (m, 4H, Hₐ), 4.94 (dd, 1H, J₃₋₂=1.2 Hz, J₃₋₄=5.1 Hz, H₃); 4.73 (d, 1H, J₁₋₂=2.2 Hz, H₁); 4.82, 4.90 (2 m, 2H, H₆); 7.18-7.32 (m, 15H, H_{c,d,e}).

¹³C NMR (100.62 MHz, CDCl₃) δ (ppm): 55.52 (—OCH₃); 72.27, 72.91 and 74.66 (3Cₐ); 74.34 (C₃); 76.08 (C₂); 79.18 (C₄); 82.34 (C₆); 99.43 (C₁); 127.59-129.11 (C_{c,d,e}); 137.73, 138.02, 140.12 (3C_{b} and C₅).

### 4) SYNTHESIS OF (3R,4R,5S,6R)-3,4,5-TRIS(BENZYLOXY)TETRAHYDRO-6-METHOXYPYRAN-2-ONE (Compound 4i)

1.13 g of (3R,4R,5S,6R)-3,4,5-tris(benzyloxy)tetrahydro-6-methoxypyr-2-ene (2.53 mmol, 1 eq.) are placed in solution in 500 ml of tBuOH, before adding 1.3 g of K₂CO₃ (8.36 mmol, 3.3 eq.) dissolved beforehand in 100 ml of water. 0.12 g of KMnO₄ (0.76 mmol, 0.3 eq.) in solution in 100 ml of water and 406 mg of NaIO₄ (1.90 mmol, 0.75 eq.) in solution in 100 ml of water are then added. After stirring for 1 h at ambient temperature, the solution is extracted with CH₂Cl₂, and the organic phase is dried, concentrated and purified on a silica column (5/5 v/v EtOAc/PE). The product is obtained in the form of a pale yellow oil (1.07 g, 94%).

Rf: 0.55 (6/4 v/v EtOAc/PE).

MS (ESI⁺/MeOH): m/z 471.18 [M+Na]⁺.

¹H NMR (400.13 MHz, CDCl₃) δ (ppm): 3.54 (s, 3H, —OCH₃); 3.73 (dd, 1H, J₂₋₁=2.7 Hz, J₂₋₃=8.1 Hz, H₂); 4.01 (m, 2H, H₄ and 1Hₐ); 4.15 (d, 2H, J=10.4 Hz, Hₐ); 4.96 (dd, 1H, J₃₋₂=1.0 Hz, J₃₋₄=5.0 Hz, H₃); 4.76 (d, 1H, J₁₋₂=2.6 Hz, H₁); 7.20-7.29 (m, 15H, H_{c,d,e}).

¹³C NMR (100.62 MHz, CDCl₃) δ (ppm): 55.51 (—OCH₃); 72.27, 72.91 and 74.66 (3Cₐ); 74.17 (C₅); 74.36 (C₃); 76.00 (C₂); 79.15 (C₄); 99.42 (C₁); 127.61-128.41 (C_{c,d,e}); 137.73, 138.02 (3C_{b}).

### 5) SYNTHESIS OF (3R,4R,5S,6R)-3,4,5-TRIS(BENZYLOXY)TETRAHYDRO-6-METHOXYPYRAN-2-OL (Compound 5i)

1.07 g of (3R,4R,5S,6R)-3,4,5-tris(benzyloxy)tetrahydro-6-methoxypyran-2-one (2.39 mmol, 1 eq.) are reacted with 180 mg of NaBH₄ (4.78 mmol, 2 eq.) in 25 ml of MeOH. After stirring for 16 hours at ambient temperature, 2 ml of water are added and the solution is then concentrated. Silica gel column chromatography (5/5 v/v EtOAc/PE) makes it possible to obtain the product in the form of a yellow oil (1 g, 94%).

Rf: 0.55 (5/5 v/v EtOAc/PE).

MS (ESI⁺/MeOH): m/z 473.67 [M+Na]⁺.

¹H NMR (400.13 MHz, CDCl₃) δ (ppm): 3.49 (s, 3H, —OCH₃); 3.60 (dd, 1H, J₂₋₁=1.2 Hz, J₂₋₃=6.8 Hz, H₂); 3.82 (m, 2H, H₄ and 1Hₐ); 4.00 (d, 1H, J=10.4 Hz, 1Hₐ); 4.10 (dd, 1H, J₃₋₂=1.0 Hz, J₃₋₄=5.0 Hz, H₃); 4.31 (t, 1H, J₅₋₄=J₅₋₆=6.2 Hz, H₅); 4.38-4.78 (m, 4H, Hₐ); 4.77 (d, 1H, J₁₋₂=2.6 Hz, H₁); 7.18-7.31 (m, 15H, H_{c,d,e}).

¹³C NMR (100.62 MHz, CDCl₃) δ (ppm): 57.40 (—OCH₃); 73.60, 74.23, 74.86 (3Cₐ); 76.65 (C₃); 78.61 (C₂); 79.12 (C₄); 100.56 (C₁); 127.97-128.64 (C_{c,d,e}); 137.14, 137.89 (3C_{b}).

### 6) SYNTHESIS OF (3R,4R,5S,6R)-3,4,5-TRIS(BENZYLOXY)TETRAHYDRO-6-OXYDIISOPROPYLPHOSPHONYLMETHOXYPYRAN (Compound 6i)

640 mg of (3R,4R,5S,6R)-3,4,5-tris(benzyloxy)tetrahydro-6-methoxypyran-2-ol (1.43 mmol, 1 eq.) are dissolved in 6.4 ml of DMF so as to form a very viscous solution. 14 mg of LiI (0.10 mmol, 0.07 eq.) and 241 mg of tBuOK (2.14 mmol, 1.5 eq.) are then added, and the solution becomes liquid. Under argon, 482 mg of phosphonate (1.86 mmol, 1.3 eq.) dissolved beforehand in 3.2 ml of DMF are added to the mixture dropwise. The reaction is left to stir for 10 minutes at ambient temperature and then heated for 3 hours at 60° C. After cooling, the mixture is diluted in EtOAc and washed with a saturated solution of NaCl. The organic phase is dried over Na₂SO₄, filtered, concentrated and purified on a silica column (8/2 v/v EtOAc/PE) to give a colorless oil (698 mg, 78%).

Rf: 0.45 (8/2 v/v EtOAc/PE).

MS (ESI⁺/MeOH): m/z 651.90 [M+Na]⁺.

1H NMR (400.13 MHz, CDCl₃) δ (ppm): 1.35 (d, 8H, J₈₋₇=6.0 Hz, H₈); 3.22 (s, 2H, H₆); 3.55 (s, 3H, —OCH₃); 3.66 (dd, 1H, J₂₋₁=1.2 Hz, J₂₋₃=6.4 Hz, H₂); 3.89 (m, 2H, H₄ and 1Hₐ); 4.06 (d, 2H, J=10.4 Hz, Hₐ); 4.16 (dd, 1H, J₃₋₂=1.2 Hz, J₃₋₄=6.0 Hz, H₃); 4.37 (t, 1H, J₅₋₄=J₅₋₆=6.2 Hz, H₅); 4.44-4.84 (m, 6H, Hₐ, H₁ and H₇); 7.26-7.36 (m, 15H, H_{c,d,e}).

¹³C NMR (100.62 MHz, CDCl₃) δ (ppm): 17.99 (C₆); 23.78, 23.83, 23.97, 24.01 (C₈); 56.94 (—OCH₃); 69.47, 71.95 and 72.67 (3Cₐ); 72.10 and 72.17 (C₇); 73.12 (C₅); 75.27 (C₃); 75.92 (C₂); 77.17 (C₄); 102.86 (C₁); 127.53-128.46 (C_{c,d,e}); 137.23, 138.35 (3C_{b}).

### 7) SYNTHESIS OF (3R,4R,5S,6R)-3,4,5-TRIS(BENZYLOXY)TETRAHYDRO-6-OXYPHOSPHONYLMETHOXYPYRAN (Compound 7i)

700 mg of (3R,4R,5S,6R)-3,4,5-tris(benzyloxy)tetrahydro-6-oxydiisopropylphosphonyl-methoxypyran (1.12 mmol, 1 eq.) are reacted in 20 ml of CH₂Cl₂ with 900 μl of pyridine (11.2 mmol, eq.) and 813 μl of TMSBr (5.59 mmol, 5 eq.). After stirring for 16 hours at ambient temperature, the mixture is diluted in EtOAc, and then washed with water. The organic phase is dried and concentrated. The transparent oil obtained is pure enough to be reused directly in reaction. The protected phosphonate dissolved in 30 ml of a 1/1 v/v MeOH/THF mixture, degassed beforehand under argon, is reacted with Pd/C under an H₂ atmosphere. After 18 hours of reaction, the solution is filtered and then concentrated. The product is obtained in the form of a white oil (43% over the 2 steps).

Rf: 0.15 (5/5 v/v isopropanol/NH₄OH).

MS (ESI⁺/MeOH): m/z 277.64 [M+Na]⁺.

¹H NMR (400.13 MHz, D₂O) δ (ppm): 3.25 (s, 3H, —OCH₃); 3.48 (m, 2H, H₄ and H₅); 3.60 (m, 2H, H₃ and H₆ₐ); 3.76 (m, 1H, H_{6b}); 3.77 (dd, 1H, J₂₋₁=1.8 Hz, J₂₋₃=3.4 Hz, H₂); 4.61 (d, 1H, J₁₋₂=2.0 Hz, H₁).

¹³C NMR (100.62 MHz, D₂O) δ (ppm): 54.61 (—OCH₃); 60.86 (C₆); 66.67 (C₄); 69.82 (C₂); 70.44 (C₃); 72.45 (C₅); 100.76 (C₁).

### Biological Results:

A—On Rat Aortic Rings

The biological effects of the compounds of the invention on angiogenesis were tested according to a technique well known to those skilled in the art: the rat aortic ring technique (Nicosia et al., M. Am. J. Pathol., 1994 November; 145(5): 1023-9). This approach is fast and consists in placing a rat aortic ring one millimeter thick in a three-dimensional culture system formed from a network of collagen type 1. The aortas are obtained from Sprague-Dawley rats and cultured for 9 to 11 days in the presence or absence of the test compounds. The angiogenesis is then assessed by evaluating the number, the size and the organization of the vascular buds progressing in the collagen lattice.

The histograms of FIGS. 12a and 12b represent the in vitro neo-angiogenic effect (Nicosia model) of various compounds of the invention:
Control: nontreated aorta,
Compound III (compound 4f): aorta treated with a compound of formula (III) in which Z is —X—HP(O)OH, X is an oxygen atom and R₂ is a methyl group (synthesized according to example 4),
Compound II: aorta treated with a compound of formula (II) in which

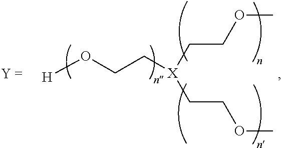

X is an oxygen atom, n=1, n′=2 and n″=0, and R₁═R′₁=—N₃,
Compound III (compound 7e): aorta treated with a compound of formula (III) in which Z is —CH₂—B(OH)₂ and R₂ is a methyl group (synthesized according to example 3),
Compound I: aorta treated with a compound of formula (I) in which A is a gold nanoparticle, R₁=—COOH and m=5, and
Reference: aorta treated with sunitinib: Sutent® (sold by Pfizer) corresponding to the formula:

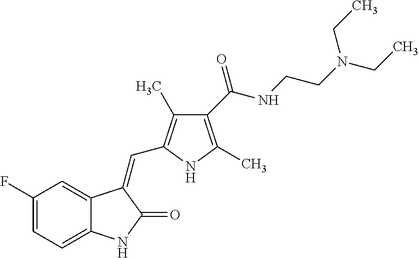 [SMILES: CCN(CC)CCNC(=O)c1c(C)[nH]c(/C=C2\C(=O)Nc3ccc(F)cc32)c1C]

B—On Endothelial Cells

Human dermal endothelial cells (15 000/well) are treated with various compounds corresponding to the definition of the invention for 48 hours.

At the end of this treatment, the cell number is estimated by means of the succinate dehydrogenase activity (MTT test, Mosmann T. J., Immunol. Methods, 1983 Dec. 16; 65(1-2): 55-63). The results show that the treatment of the cells with the compounds of the invention at three different doses (10⁻² mol·l⁻¹, 10⁻⁴ mol·l⁻¹ and 10⁻⁶ mol·l⁻¹) has no cytotoxic effect on the endothelial cells.

The histogram of FIG. 13 represents the cytotoxic effect of various compounds:
Control: nontreated cells,
Compound III (compound 4f): cells treated with a compound of formula (III) in which Z is —X—HP(O)OH, X is an oxygen atom and R₂ is a methyl group (synthesized according to example 4),
Compound II: cells treated with a compound of formula (II) in which:

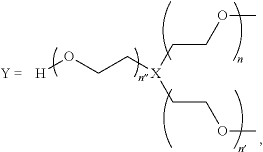

X is an oxygen atom, n=1, n′=2, n″=0, and R₁═R′₁=—N₃,
Compound I: cells treated with a compound of formula (I) in which A is a gold nanoparticle, R₁=—COOH and m=5, and
Compound III (compound 7e): cells treated with a compound of formula (III) in which Z is —CH₂—B(OH)₂ and R₂ is a methyl group (synthesized according to example 3).

C—On mice which have a B16 melanoma

A melanoma is induced in a mouse by subcutaneous injection of tumor cells (B16 cells).

The tumor develops within 10 days in the subcutaneous position (ventral face of the thigh). The size of the tumor is measured with a caliper rule and its volume is estimated according to the formula V=L×w×w, in which L represents the length and w represents the width. Various compounds of the invention were administered to mice having received syngenic melanoma cells (B16) at a dose of 300 mg/kg.

The various compounds tested are the following:
Control: nontreated mice,
M6P: mice treated with mannose-6-phosphate (M6P),
Compound III (compound 4f): mice treated with a compound of formula (III) in which Z is —X—HP(O)OH, X is an oxygen atom and R₂ is a methyl group (synthesized according to example 4),
Compound III (compound 7e): mice treated with a compound of formula (III) in which Z is —CH₂—B(OH)₂ and R₂ is a methyl group (synthesized according to example 3),
Compound II: cells treated with a compound of formula (II) in which

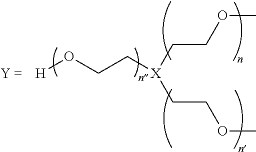

X is an oxygen atom, n=1, n′=2, n″=0, and R₂═R₁₂=—N₃,
Compound I: cells treated with a compound of formula (I) in which A is a gold nanoparticle, R₁=—COOH and m=3.

The survival rate of the mice as a function of the number of days of treatment is represented on the graph of the appended FIG. 14.

The change in tumor growth as a function of the number of days of treatment is represented on the graph of the appended FIG. 15.

A strong inhibition (65%) of tumor growth was observed in the mouse group treated with compound 7f of formula (III) of the invention, an inhibition of 40% was observed in the mouse group treated with compound 4e of formula (III) of the invention, and an inhibition of 30% was observed in the mouse group treated with the compound of formula (I) of the invention. In parallel, no mortality is observed in the mouse group treated with compound 7f of formula (III) of the invention after 18 days of treatment, in comparison with the nontreated mouse group (40% mortality).

D—Angiogenic Activity on Chorioallantoic Membrane (CAM)

The results obtained during a conventional test for studying angiogenesis in vivo, carried out on chicken embryo chorioallantoic membrane (CAM), can be observed on the photographs of the appended FIG. 16.

The various compounds tested are the following:
Control: nontreated mice,
Reference: mice treated with Sutent®,
M6P: mice treated with mannose-6-phosphate (M6P),
Compound I: cells treated with a compound of formula (I) in which A is a gold nanoparticle, R₁=—N₃ and m=5,
Compound IIₐ: cells treated with a compound of formula (II) in which

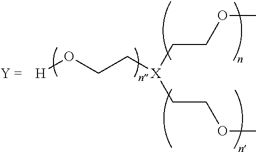

X is an oxygen atom, n=1, n′=2, n″=0, and R₁═R′₁=—N₃,
Compound II_{b}: cells treated with a compound of formula (II) (compound of example 7) in which

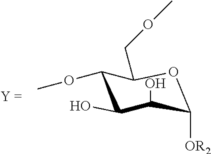

R₁═R′₁=—N₃, and R₂ is a methyl group,
Compound III (compound 4f): mice treated with a compound of formula (III) in which Z is —X—HP(O)OH, X is an oxygen atom and R₂ is a methyl group (synthesized according to example 4),
Compound III (compound 7e): mice treated with a compound of formula (III) in which Z is —CH₂—B(OH)₂ and R₂ is a methyl group (synthesized according to example 3).

The compounds of the invention exhibit a strong inhibitory effect, and also considerably fewer side effects compared with those of the reference. The compounds of the invention also exhibit anti-angiogenic activity greater than that of M6P.

1. A mannopyranoside-derived compound or pharmaceutically acceptable salts thereof, characterized in that it corresponds to one of the following formulae: 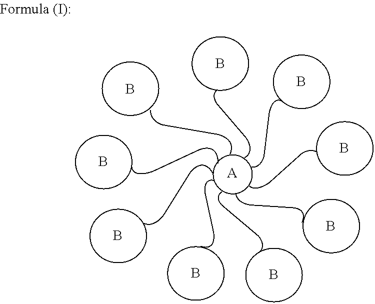
in which:
A is a silica nanoparticle or a metal nanoparticle chosen from the elements of columns (IB), (IIB), (IIIB), (IVB), (VB), (VIB), (VIIB) or (VIIIB) of Mendeleev's periodic table, and
B is a group carrying a mannopyranoside function corresponding to the following structure: 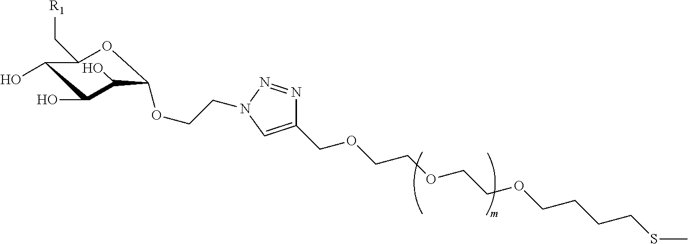
in which m is an integer between 0 and 10, and preferably m=3, 4, 5 or 6,
the B groups being bonded to the nanoparticle A via the sulfur atom, and the number of B groups bonded to the nanoparticle A being between 100 and 1000, and preferably between 400 and 600, 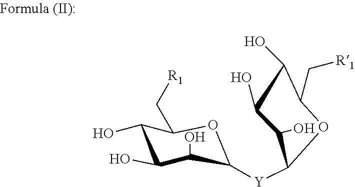
in which Y represents one of the following groups: 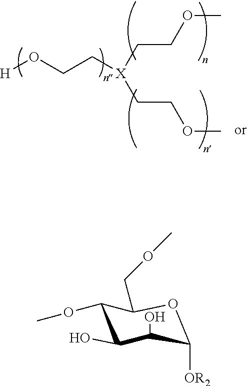
with:
n, n′ and n″ being integers between 1 and 12, and preferably between 1 and 6, and
n″ being equal to 0 when X represents an oxygen atom, 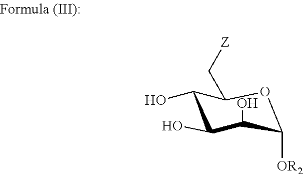
in which Z represents one of the following groups: 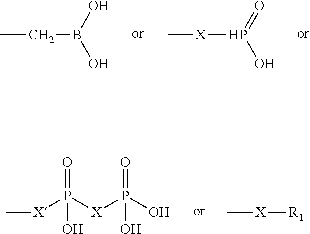
in which:
the R₁ and R′₁ radicals, which may be identical or different, represent a radical selected from —O—PO₃H₂, —N₃, —CH₂—PO₃H₂, —CH₂—COOH, —SO₃H₂, —OPHO₂H, —CH₂—B(OH)₂, —X—PHO₂H, X′—PO₂H—X—PO₃H₂, and preferably —CH₂—COOH and —N₃,
the R₂ radical represents a linear or branched C₁-C₁₂, and preferably C₁-C₄, alkyl chain; a linear or branched C₁-C₁₂, and preferably C₁-C₄, alkyl chain carrying at least one —OH, —NH₂, —SH, —COOH, —N₃ or —NO₂ group; a saturated or unsaturated C₃-C₆ hydrocarbon-based ring; a saturated or unsaturated C₃-C₁₀ hydrocarbon-based ring carrying at least one —OH, —NH₂, —SH, —COOH, —N₃, —NO₂ or C₁-C₄ alkyl group; a saturated or unsaturated heterocycle comprising at least one heteroatom chosen from oxygen, nitrogen or sulfur atoms; a —(CH₂—CH₂—O)_{y}—H radical, in which y is between 1 and 12, and preferably between 1 and 6, and
the X and X′ groups, which may be identical or different, are chosen from: N, O, S and a C₁-C₄ alkyl chain, the X and X′ groups preferably being oxygen atoms.

2. The compound as claimed in claim 1, characterized in that R₂ represents a C₁-C₄ alkyl chain chosen from methyl, ethyl, n-propyl, isopropyl, n-butyl, tert-butyl, isobutyl and n-hexyl radicals, and preferably the methyl radical.

3. The compound as claimed in claim 1, characterized in that R₂ represents a saturated hydrocarbon-based ring chosen from cyclopropane, cyclobutane, cyclopentane and cyclohexane.

4. The compound as claimed in claim 1, characterized in that R₂ represents an unsaturated hydrocarbon-based ring or a saturated heterocycle chosen from phenyl, oxadiazole, triazole, oxazole, isoxazole, imidazole, thiadiazole, pyrrole, tetrazole, furan, thiophene, pyrazole, pyrazoline, pyrazolidine, thiazole, isothiazole, pyridine, pyrimidine, piperidine, pyran, pyrazine, pyridazine, indole, indazole, benzoxazole, naphthalene, quinoline, quinoxaline, quinazoline, anthracene and acridine rings, and preferably phenyl rings.

5. The compound as claimed in one of claims 1 to 4, characterized in that the nanoparticles A of the compound of formula (I) are chosen from gold, iron and cobalt nanoparticles.

6. The compound as claimed in one of claims 1 to 5, characterized in that the nanoparticles A have a diameter of between 2 and 10 nm, and preferably between 4 and 8 nm.

7. A process for preparing a mannopyranoside-derived compound as claimed in one of claims 1 to 6, characterized in that it comprises at least the following steps:
(i) a step of halogenation between a compound of formula (I′), (II′) or (III′) carrying at least one primary alcohol function, by reaction with a dihalogen/phosphine or N-halosuccinimide/-phosphine mixture, said compounds (I′), (II′) or (III′) corresponding to the following formulae: 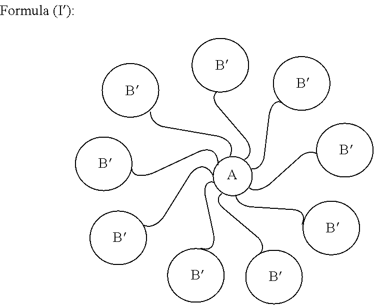
in which A is as defined in claims 1 to 6, and B′ is a group corresponding to the following structure: 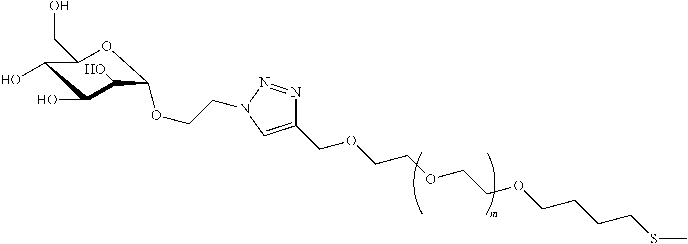
in which m is as defined in claims 1 to 6,
the B′ groups being bonded to the nanoparticle A by the sulfur atom, and the number of B′ groups bonded to the nanoparticle A being between 100 and 1000, and preferably between 400 and 600, 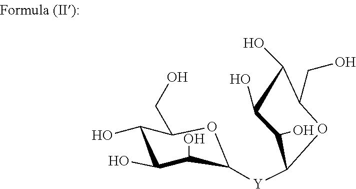
in which Y, n, n′ and n″ are as defined in claims 1 to 6, 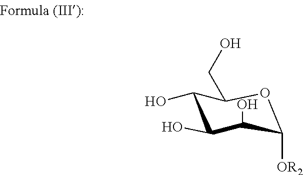
the radical R₂ and the X and X′ groups being as defined in claims 1 to 6,
(ii) a step of nucleophilic substitution of the halogenated compounds obtained in step (i), by reaction with a nucleophilic reagent carrying an R₁ and/or R′₁ radical, so as to obtain the compounds of formula (I), (II) or (III) as defined in claims 1 to 6.

8. The process as claimed in claim 7, characterized in that step (i) is carried out with a diiodine/triphenylphosphine mixture, in the presence of imidazole.

9. The mannopyranoside-derived compound as claimed in one of claims 1 to 6, for use as a medicament.

10. The compound as claimed in claim 9, for use as a medicament intended for the prevention and/or treatment of diseases dependent on an inhibition of angiogenesis.

11. The compound as claimed in claim 10, for use as a medicament intended for the treatment of cancer diseases, diabetic blindness, macular degeneration, rheumatoid arthritis and psoriasis.

12. The compound as claimed in claim 11, for use as a medicament intended for the treatment of cancer diseases.

13. A pharmaceutical composition, characterized in that it comprises, as active ingredient, at least one mannopyranoside-derived compound as defined in any one of claims 1 to 6, and at least one pharmaceutically acceptable excipient.

14. The pharmaceutical composition as claimed in claim 13, characterized in that it comprises one or more antitumor active ingredients chosen from doxorubicin, etoposide, fluorouracil, melphalan, cyclophosphamide, bleomycin, vinblastin, mitomycin, lomustine (CCNU), taxotere, taxol, methotrexate and cisplatinum.

15. An implantable medical device, characterized in that it is surface-treated with at least one mannopyranoside-derived compound as defined in one of claims 1 to 6, it being possible for said device to be chosen from prostheses, and more particularly vascular, urethral and biliary stents.
